(19) United States
(12) Patent Application Publication
(10) Pub. No.: US 2011/0219462 A1
(43) Pub. Date: Sep. 8, 2011

(54) G-Protein Coupled Receptor 30 (GPR30) transgenic animals as a model for cardiovascular diseases

(75) Inventors: Martina Delbeck, Heiligenhaus (DE); Stefan Golz, Mulheim an der Ruhr (DE); Stefan Schafer, Goch (DE); Christiane Otto, Berlin (DE)

(73) Assignee: BAYER SCHERING PHARMA AKTIENGESELLSCHAFT, Berlin (DE)

(21) Appl. No.: 13/054,509

(22) PCT Filed: Jul. 2, 2009
(86) PCT No.: PCT/EP09/04769

§ 371 Date: Apr. 8, 2011

(30) Foreign Application Priority Data
Jul. 15, 2008 (EP) 08012714.5

(51) Int. Cl. A01K 67/00; A61K 9/00; C12N 5/07; G01N 33/567

(52) U.S. Cl. 800/3; 800/13; 800/9; 800/14; 435/325; 435/7.21

(57) ABSTRACT

The present invention relates to use of the GPR30 gene for diagnosis and treatment of cardiovascular disorders, especially cardiomyopathy. The present invention also relates to a GPR30 deficient animal model, more specifically to a mouse in which the GPR30 gene is disrupted and which exhibits a cardiomyopathy, a tissue and a cell of the mouse and a process of producing the same. The present invention further relates to use of said knockout mouse as a model of cardiovascular diseases, especially cardiomyopathy, and a method of screening a compound useful for the prevention and/or treatment of cardiovascular diseases, especially cardiomyopathy, using the knockout mouse.

### TECHNICAL FIELD OF THE INVENTION

The present invention relates to use of the GPR30 gene for diagnosis and treatment of cardiovascular disorders, especially cardiomyopathy. The present invention also relates to a GPR30 knockout mouse, more specifically to a mouse in which the GPR30 gene is disrupted and which exhibits a cardiomyopathy, a tissue and a cell of the mouse and a process of producing the same.

The present invention further relates to use of said knockout mouse as a model of cardiovascular diseases, especially cardiomyopathy, and a method of screening a compound useful for the prevention and/or treatment of cardiovascular diseases, especially cardiomyopathy, using the knockout mouse.

### BACKGROUND OF THE INVENTION

### Animal Models of Cardiomyopathy

Cardiomyopathy is a growing public health problem. Many of these people suffer from heart failure and every year cardiomyopathy is a contributing factor in nearly a quarter million deaths. No treatment, except for heart transplantation, can completely suppress the progression of the pathology. Therefore, a novel therapeutic option for cardiomyopathy has been desired. In order to find a new drug for the treatment of cardiomyopathy, a screening using an animal model of cardiomyopathy is of importance. At present, the Bio 14.6 hamster, reported to be caused by large deletion in the exon 1 and the promoter region of the delta-sarcoglycan gene, is one of the most widely used models of cardiomyopathy. Furthermore cardiac hypertrophy or heart failure has been induced in mice, rats, dogs, rabbits and many other animals by various manipulations such as coronary artery ligation, drugs, pressure and/or volume overload and chronic rapid pacing [Date at al. (2000)]. There are also some transgenic mice which exhibit cardiomyopathy [Zhanq et al. (2001), [Kubora et al. (1997)]. Nevertheless, there is a need for animal models for cardiovascular diseases, especially cardiomyopathy.

### G-Protein Coupled Receptors

GPR30 is a seven transmembrane G protein coupled receptor (GPCR) [Owman et al. (1996)], [Carmeci et al. (1997)], [Feng and Gregor (1997)], WO02061087, U.S. Pat. No. 6,489,442, U.S. Pat. No. 6,468,769].

Many medically significant biological processes are mediated by signal transduction pathways that involve G-proteins [Lefkowitz, (1991)]. The family of G-protein coupled receptors (GPCRs) includes receptors for hormones, neurotransmitters, growth factors, and viruses. Specific examples of GPCRs include receptors for such diverse agents as dopamine, calcitonine, adrenergic hormones, endotheline, cAMP, adenosine, acetylcholine, serotonine, histamine, thrombin, kinin, hormones, opsins, endothelial differentiation gene-1, rhodopsins, odorants, cytomegalovirus, G-proteins themselves, effector proteins such as phospholipase C, adenyl cyclase, and phosphodiesterase, and actuator proteins such as protein kinase A and protein kinase C.

GPCRs possess seven conserved membrane-spanning domains connecting at least eight divergent hydrophilic loops. GPCRs, also known as seven transmembrane, 7TM, receptors, have been characterized as including these seven conserved hydrophobic stretches of about 20 to 30 amino acids, connecting at least eight divergent hydrophilic loops. Most GPCRs have single conserved cysteine residues in each of the first two extracellular loops, which form disulfide bonds that are believed to stabilize functional protein structure. The seven transmembrane regions are designated as TM1, TM2, TM3, TM4, TM5, TM6, and TM7. TM3 is being implicated with signal transduction. Phosphorylation and lipidation (palmitylation or farnesylation) of cysteine residues can influence signal transduction of some GPCRs. Most GPCRs contain potential phosphorylation sites within the third cytoplasmic loop and/or the carboxy terminus. For several GPCRs, such as the beta-adrenergic receptor, phosphorylation by protein kinase A and/or specific receptor kinases mediates receptor desensitization.

For some receptors, the ligand binding sites of GPCRs are believed to comprise hydrophilic sockets formed by several GPCR transmembrane domains. The hydrophilic sockets are surrounded by hydrophobic residues of the GPCRs. The hydrophilic side of each GPCR transmembrane helix is postulated to face inward and form a polar ligand binding site. TM3 is being implicated with several GPCRs as having a ligand binding site, such as the TM3 aspartate residue. TM5 serines, a TM6 asparagine, and TM6 or TM7 phenylalanines or tyrosines also are implicated in ligand binding.

GPCRs are coupled inside the cell by heterotrimeric G-proteins to various intracellular enzymes, ion channels, and transporters. Different G-protein alpha-subunits preferentially stimulate particular effectors to modulate various biological functions in a cell. Phosphorylation of cytoplasmic residues of GPCRs is an important mechanism for the regulation of some GPCRs. For example, in one form of signal transduction, the effect of hormone binding is the activation of the enzyme, adenylate cyclase, inside the cell. Enzyme activation by hormones is dependent on the presence of the nucleotide GTP. GTP also influences hormone binding. A G-protein connects the hormone receptor to adenylate cyclase. G-protein exchanges GTP for bound GDP when activated by a hormone receptor. The GTP-carrying form then binds to activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

7TM GPCRs consist of more than 800 human members, of which more than 300 are likely to be olfactory receptors. Of the remaining receptors, more than 100 remain as orphan receptors, i.e., cloned receptors for which no ligand is known [Civelli (2005)]. Over the past 15 years, nearly 350 therapeutic agents targeting 7TM receptors have been successfully introduced into the market. This indicates that these receptors have an established, proven history as therapeutic targets. Clearly, there is a need for identification and characterization of further receptors which can play a role in preventing, ameliorating, or correcting dysfunctions or diseases including, but not limited to, cardiovascular diseases.

### GPR30

The G protein-coupled (heptahelix) membrane receptors receive chemical signals in cell communication in both CNS and peripheral tissues. This receptor type is recognized by many chemoattractant peptides, the model substance being interleukin-8 (IL8), and heptahelix receptors are now recognized among cluster-determinant antigens in immune cells, i.e., CDw78 and CD97. These receptors may also be involved in other functional mechanisms, such as viral pathogenesis. For example, the human cytomegalovirus shows structural homology with heptahelix receptors and encodes a functional chemokine receptor, and Herpesvirus saimiri exerts ‘molecular piracy’ of the IL8B [Ahuja et al. (1993)] receptor. Herpesvirus saimiri is closely related to the B-lymphotropic Epstein-Barr virus, which has been implicated in several human malignancies such as Burkitt lymphoma [Schuster et al. (1992)] which expresses BLR1 [Dobner et al. (1992)], a heptahelix receptor.

Owman et al. [Owman et al. (1996)] performed PCR of template DNA from human B-cell lymphoblasts using degenerate primers to G protein-coupled receptors. They identified a full-length cDNA encoding a 375-amino acid protein, which they termed CMKRL2 (GPR30).

Using the technique of differential cDNA library screening to identify genes overexpressed in estrogen receptor (ER)-positive breast carcinoma cell lines compared to ER-negative cell lines, Carmeci et al. [Carmeci et al. (1997)] isolated a CMKRL2 cDNA and designated it GPR30.

Sequence analysis determined that the clone shared significant homology to G protein-coupled receptors. This receptor was abundantly expressed in 3 ER-positive breast carcinoma cell lines. Expression was absent or minimal in 3 ER-negative breast carcinoma cell lines. It was ubiquitously expressed in all human tissues examined, but was most abundant in placenta. In 11 primary breast carcinomas, it was detected in all 4 ER-positive tumors and in only 1 of 7 ER-negative tumors. The pattern of expression of the gene indicated that the receptor may be involved in physiologic responses specific to hormonally responsive tissues.

Using PCR with degenerate primers to identify novel members of the peptide-binding G protein-coupled receptor (GPCR) family, Feng and Gregor [Feng and Gregor (1997)] also cloned CMKRL2.

At the same time O'Dowd et al. [O'Dowd et al. (1997)] cloned GPR30 using PCR with degenerate oligonucleotides based on GPR1. The amino acid sequence encoded by GPR30 showed highest identity with members of the chemoattractant receptor family, namely, formylpeptide receptor FPRL1 (˜32% overall identity) and formylpeptide-like receptor FPRL2 (˜32% overall identity), and with chemokine receptor CXCR1 (˜29% overall identity), suggesting that the endogenous ligand may be a chemokine.

Owman et al. [Owman et al. (1996)] mapped the CMKRL2 gene to 7p22 by fluorescence in situ hybridization. Based upon PCR analysis in somatic hybrid cell lines, Carmeci et al. [Carmeci et al. (1997)] mapped the gene encoding the GPR30 gene to 7p22.

GPR30 was described as an estrogen binding GPCR by Thomas et al. [Thomas et al.,(2005)].

GPR30 is published in patents W002061087, U.S. Pat. No. 6489442 and U.S. Pat. No. 6468769.

Other names which have been used for GPR30 include CMKRL2, CEPR, DRY12, FEG-1, LERGU, LyGPR, LERGU2, GPCR-Br and MGC99678.

The nucleotide sequence of GPR30 is accessible in the databases by the accession number Y08162 (human) and AK030375 (mouse). The sequences are given in SEQ ID NO:1 (human) and SEQ ID NO:2 (mouse). The amino acid sequence of GPR30 depicted in SEQ ID NO:3 (human) and SEQ ID NO:4 (mouse).

### SUMMARY OF THE INVENTION

The invention relates to use of GPR30 as a target for diagnosis and treatment of cardiovascular disorders, especially cardiomyopathy. The invention also relates to novel methods of screening for therapeutic agents for the treatment of cardiovascular diseases, especially cardiomyopathy.

The invention also relates to pharmaceutical compositions for the treatment of cardiovascular diseases, especially cardiomyopathy, comprising a GPR30 polypeptide, a GPR30 polynucleotide, or regulators of GPR30 or modulators of GPR30 activity. The invention further comprises methods of diagnosing cardiovascular diseases, especially cardiomyopathy.

The present invention also relates to a GPR30 knockout mouse which exhibits a cardiomyopathy. The present invention further relates to use of said knockout mouse as a model mouse of cardiovascular diseases, especially cardiomyopathy and a method of screening compounds useful for the prevention and/or treatment of cardiovascular diseases using said knockout mouse.

### BRIEF DESCRIPTION OF THE DRAWINGS

FIG. 1 shows the heart weight related to the tibia length (mg/mm) [a] of GPR30 knockout mice (1) versus wildtype mice (2) [b] (mean+Standard Error of the Mean (SEM), n=12-19 per group, *, p<0.05 vs wildtype mice).

FIG. 2 shows the left ventricular systolic pressure (LVPsys, mmHg) [a] of GPR30 knockout mice (1) versus wildtype mice (2) [b] (mean+SEM, n=8-11 per group, *, p<0.05 vs wildtype mice).

FIG. 3 shows the left ventricular dPdtₘₐₓ (mmHg/s) [a] of GPR30 knockout mice (1) versus wildtype mice (2) [b] (mean+SEM, n=8-11 per group, *, p<0.05 vs wildtype mice).

FIG. 4 shows the left ventricular enddiastolic pressure (LVEDP, mmHg) [a] of GPR30 knockout mice (1) versus wildtype mice (2) [b] (mean+SEM, n=8-11 per group, *, p<0.05 vs wildtype mice).

FIG. 5 shows left ventricular tau (s) [a] of GPR30 knockout mice (1) versus wildtype mice (2) [b] (mean+SEM, n=8-11 per group, *, p<0.05 vs wildtype mice).

FIG. 6 shows the left ventricular dPdtₘᵢₙ (mmHg/s) [a] of GPR30 knockout mice (1) versus wildtype mice (2) [b] (mean+SEM, n=8-11 per group, *, p<0.05 vs wildtype mice).

FIG. 7 shows the heart rate (beats per minute) [a] of GPR30 knockout mice (1) versus wildtype mice (2) [b] (mean+SEM, n=12 per group, *, p<0.05 vs wildtype mice).

FIG. 8 shows the RR interval (ms) [a] of GPR30 knockout mice (1) versus wildtype mice (2) [b] (mean+SEM, n=12 per group, *, p<0.05 vs wildtype mice).

FIG. 9 shows the running distance per night (km) [a] of GPR30 knockout mice (1) versus wildtype mice (2) over a period of 21 days after 7 days running-in period [b] (mean+SEM, n=5 per group, *, p<0.05 vs wildtype mice).

FIG. 10 shows the top speed per night (km/h) [a] of GPR30 knockout mice (1) versus wildtype mice (2) over a period of 21 days after 7 days running-in period [b] (mean+SEM, n=5 per group, *, p<0.05 vs wildtype mice).

FIG. 11 shows the average speed per night (km/h) [a] of GPR30 knockout mice (1) versus wildtype mice (2) over a period of 21 days after 7 days running-in period [b] (mean+SEM, n=5 per group, *, p<0.05 vs wildtype mice).

FIG. 12 shows the longest running period per night (m) [a] of GPR30 knockout mice (1) versus wildtype mice (2) over a period of 21 days after 7 days running-in period [b] (mean+SEM, n=5 per group, *, p<0.05 vs wildtype mice).

FIG. 13 shows the nucleotide sequence of human GPR30

FIG. 14 shows the nucleotide sequence of mouse GPR30

FIG. 15 shows the aminoacid sequence of human GPR30

FIG. 16 shows the aminoacid sequence of mouse GPR30

FIG. 17 shows the relative expression of Triadin (Trdn) [a] in right ventricle (1), left ventricle (2), septum (3) of a GPR30 knock out mouse and right ventricle (4), left ventricle (5), septum (6) of a wildtype mouse [b] determined by a microarray experiment.

FIG. 18 shows the relative expression of TIMP4 [a] in right ventricle (1), left ventricle (2), septum (3) of a GPR30 knock out mouse and right ventricle (4), left ventricle (5), septum (6) of a wildtype mouse [b] determined by a microarray experiment.

FIG. 19 shows the relative expression of interleukin 24 (IL24) [a] in right ventricle (1), left ventricle (2), septum (3) of a GPR30 knock out mouse and right ventricle (4), left ventricle (5), septum (6) of a wildtype mouse [b] determined by a microarray experiment.

FIG. 20 shows the relative expression of MMP12 [a] in right ventricle (1), left ventricle (2), septum (3) of a GPR30 knock out mouse and right ventricle (4), left ventricle (5), septum (6) of a wildtype mouse [b] determined by a microarray experiment.

FIG. 21 shows the relative expression of BTG2 [a] in right ventricle (1), left ventricle (2), septum (3) of a GPR30 knock out mouse and right ventricle (4), left ventricle (5), septum (6) of a wildtype mouse [b] determined by a microarray experiment.

FIG. 22 shows the relative expression of Thrombospondin (Thbs) [a] in right ventricle (1), left ventricle (2), septum (3) of a GPR30 knock out mouse and right ventricle (4), left ventricle (5), septum (6) of a wildtype mouse [b] determined by a microarray experiment.

FIG. 23 shows the relative expression of NR4a1 [a] in right ventricle (1), left ventricle (2), septum (3) of a GPR30 knock out mouse and right ventricle (4), left ventricle (5), septum (6) of a wildtype mouse [b] determined by a microarray experiment.

FIG. 24 shows a Cartoon model of junctin, triadin, calsequestrin (CSQ), and ryanodine receptor protein interactions in junctional SR from heart [Zang et al. (1997)].

FIG. 25 shows the relative expression of ATF3 in left ventricle wt (1), left ventricle KO (2) determined by a microarray experiment.

FIG. 26 shows the relative expression of ADAMTS1 in left ventricle wt (1), left ventricle KO (2) determined by a microarray experiment.

FIG. 27 shows the relative expression of KAP in left ventricle wt (1), left ventricle KO (2) determined by a microarray experiment.

FIG. 28 shows the relative expression of SLC27a2 in left ventricle wt (1), left ventricle KO (2) determined by a microarray experiment.

### DETAILED DESCRIPTION OF THE INVENTION

GPCRs are a conserved family of seven transmembrane receptors that is one of the largest classes of receptors to be targeted for drug therapy. Among an estimated 200 cardiac GPCRs, drugs targeting adrenergic and angiotensin GPCR signaling pathways alone account for the majority of prescriptions for cardiovascular diseases (Salazar et al. (2007)]. However, there is no report to point out the relation of cardiovascular diseases, especially cardiomyopathy, to the GPCR GPR30. Therefore, this invention provides a transgenic (knockout) mouse comprising a disrupted GPR30 gene and having a cardiovascular phenotype, and relates to use of GPR30 as a target for diagnosis and treatment of cardiovascular disorders, preferably cardiomyopathy.

As used herein, a “transgenic” organism is an organism containing a defined change to its germ line, wherein the change is not ordinarily found in wildtype organism. This change can be passed on to the organism's progeny. The change to the organism's germ line can be an insertion, a substitution, or a deletion. Thus, the term “transgenic” encompasses organisms where a gene has been eliminated or disrupted so as to result in the elimination of a phenotype associated with the disrupted gene (knockout mice). The term “transgenic” also encompasses organisms containing modifications to their existing genes and organisms modified to contain exogenous genes introduced into their germ line. The term “transgenic” also encompasses animals with tissue specific modifications of a gene.

The term “disrupted gene” as used in this application refers to a gene containing an insertion, substitution, or deletion resulting in the loss of substantially all of the biological activity associated with the gene.

The term “suboptimal levels of GPR30 polypeptide” as used in the invention refers to all genetic modification of the GPR30 gene or GPR30 mRNA resulting in a reduced expression or reduced activity of the GPR30 polypeptide leading to cardiovascular defects. Genetic modifications of GPR30 mRNA are, but not limited to, modulations of expression by the means of GPR30 specific siRNA, microRNA or RNAi.

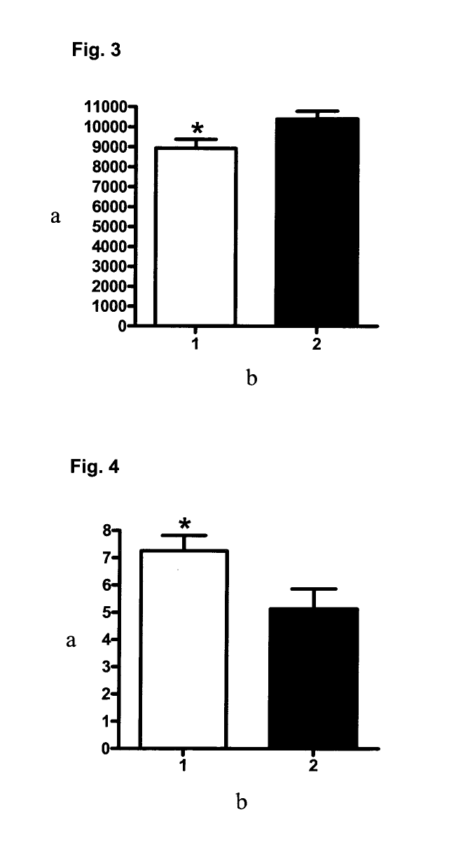

The GPR30 knockout mouse in the present invention preferably expresses cardiac dysfunction. The cardiac dysfunction as used herein includes, for example, an increase in left ventricular enddiastolic pressure (LVEDP) and left ventricular relaxation time constant (tau) as well as a decrease in cardiac contractility (LVdPdtₘₐₓ) and maximum velocity of the left ventricular pressure fall (LVdPdtₘᵢₙ) (FIGS. 3-6).

It is desired that the above-mentioned parameters with respect to the cardiac dysfunction show a statistically significant difference in comparison with that of wildtype mice.

The GPR30 knockout mouse was found to be useful as an animal model to study cardiovascular diseases, especially cardiomyopathy.

Therefore, the present invention provides a method for screening compounds using the GPR30 knockout mice as an animal model to identify compounds useful in preventing and treating cardiovascular diseases, especially cardiomyopathy. More specifically, the screening method comprises:

1) administering a test compound or a placebo to a GPR30 knockout mouse of the invention.
2) comparing placebo treatment versus a test compound in the GPR30 knockout mouse in terms of the test results to determine effectiveness of the test compound. Amelioration of one of the aforementioned defects indicates the effectiveness of a test compound.

Examples of compounds that can be screened using the method of the invention include but are not limited to rationally designed and synthetic molecules, peptides, proteins and the like, as well as tissue extract, plant extracts, animal extracts, cell culture supernatant of warm-blooded mammal and the like.

The compounds to be tested can be administered to the transgenic mouse having a disrupted GPR30 gene in a variety of ways, for example parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Oral compositions generally include an inert diluent or an edible carrier.

### Expression-Analysis

### Microarray-Analysis

Nucleic acid arrays that have been used in the present invention are those that are commercially available from Affymetrix (Santa Clara, Calif.) under the brand name Mouse Genome U230 plus 2.0 Array which represents the complete coverage of the mouse Genome. Affymetrix (Santa Clara, Calif.) GeneChip technology platform which consists of high-density microarrays and tools to help process and analyze those arrays, including standardized assays and reagents, instrumentation, and data management and analysis tools.

GeneChip microarrays consist of small DNA fragments (referred to as probes), chemically synthesized at specific locations on a coated quartz surface. By extracting and labeling nucleic acids from experimental samples, and then hybridizing those prepared samples to the array, the amount of label can be monitored enabling a measurement of gene regulation.

The GeneChip human genome arrays include a set of mouse maintenance genes to facilitate the normalization and scaling of array experiments and to perform data comparison. This set of normalization genes shows consistent levels of expression over a diverse set of tissues.

### Patients Exhibiting Symptoms of Disease

A number of diseases are associated with changes in the copy number of a certain gene. For patients having symptoms of a disease, the real-time PCR method or microarray technology can be used to determine if the patient has copy number alterations which are known to be linked with diseases that are associated with the symptoms the patient has.

### Regulated Genes

### Trdn

### Trdn—Triadin (NM_{—}006073)

The dihydropyridine-sensitive calcium channels and ryanodine-sensitive calcium channels (RYR1) of skeletal muscle play key roles in the generation of calcium transients during excitation/contraction coupling. The coupling of the signal for calcium release between these proteins occurs at highly specialized triadic junctions that separate the T-tubule membrane and the terminal cisternae of the sarcoplasmic reticulum (SR). Triadin, a protein found in rabbit triadic junctions, is intrinsic to the terminal cisternae and is closely associated with the RYR1. By RT-PCR of human skeletal muscle RNA with primers based on the sequence of a rabbit triadin cDNA. The predicted 729-amino acid human protein shares 95% identity with rabbit triadin. Like the rabbit protein, human triadin contains a small cytoplasmic domain and a single transmembrane domain. Human triadin has a calculated pI of 9.3 and molecular mass of 82 kD.

Using the alpha-myosin heavy chain promoter to drive protein expression, Kirchhefer [Kirchhefer et al. (2001)] developed transgenic mice overexpressing Trdn1, the dominant cardiac isoform of mouse triadin, in the atrium and ventricle. Expression was elevated 5-fold and was accompanied by cardiac hypertrophy. The levels of 2 other junctional SR proteins, RYR2 and junctin, were reduced by 55% and 73%, respectively. The levels of the junctional SR Ca(2+)-binding protein, calsequestrin, and the free SR Ca(2+)-handling proteins, phospholamban and Serca2a, were unchanged. The contractile phenotype of hearts from triadin-overexpressing mice included impaired relaxation, blunted contractility with increased pressure loading, and frequency-dependent changes in myocyte shortening. Kirchhefer [Kirchhefer et al. (2001)] concluded that Trdnl plays an active role in Ca(2+) release, beyond its previously proposed structural role of anchoring calsequestrin to RYR2

The overexpression of Trdn leads to blocking of excitation-contraction coupling in rat skeletal myotubes [Rezgui et al. (2005)], increases predisposition to cellular arrhythmia in cardiac myocytes [Terentyev et al. (2005)] and heart failure.

Trdn expression is up regulated in GPR30 KO mice as shown in FIG. 17.

### TIMP4

### TIMP4—Tissue Inhibitor of Metalloproteinase 4 (NM_{—}003256)

The tissue inhibitors of metalloproteinases (TIMPs) inhibit matrix metalloproteinases (MMPs), a group of peptidases involved in degradation of the extracellular matrix. Bigg et al. (1997) demonstrated specific, high-affinity binding between TIMP4 and progelatinase A (MMP2; 120360). Binding appeared to occur mainly via the C-terminal hemopexin-like domain (C domain) of gelatinase A.

Timp4 is involved in heart remodeling [Polyakova et al. (2008)] and heart failure [Felkin et al. (2006)].

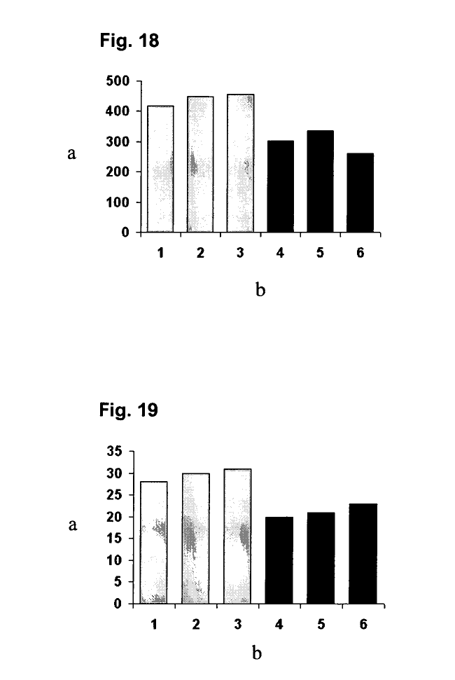

TIMP4 expression is up regulated in GPR30 KO mice as shown in FIG. 18.

### Il24

### Il24—Interleukin 24 (NM_{—}006850, NM_{—}181339)

Interleukins are a group of cytokines (secreted signaling molecules) that were first seen to be expressed by white blood cells (leukocytes, hence the -leukin) as a means of communication (inter-) The name is something of a relic though; it has since been found that interleukins are produced by a wide variety of bodily cells. The function of the immune system depends in a large part on interleukins, and rare deficiencies of a number of them have been described, all featuring autoimmune diseases or immune deficiency.

IL-24 is a cytokine belonging to the IL-10 family of cytokines that signals through two heterodimeric receptors: IL-20R1/IL-20R2 and IL-22R1/IL-20R2. This interleukin is also known as Melanoma differentiation-associated 7 (mda-7) due to its discovery as a tumour suppressing protein. IL-24 appears to control in cell survival and proliferation by inducing rapid activation of particular transcription factors called Stat-1 and Stat-3. This cytokine is predominantly released by activated monocytes, macrophages and T helper 2 (Th2) cells and acts on non-haematopoietic tissues such as skin, lung and reproductive tissues. IL-24 performs important roles in wound healing, psoriasis and cancer. Several studies have shown that cell death occurs in cancer cells/cell lines following exposure to IL-24.

Jiang [Jiang et al. (1995)] used a differentiation induction subtraction hybridization strategy to identify and clone genes involved in growth control and terminal differentiation in human cancer cells. By this approach they identified melanoma differentiation-associated gene-7 (MDA7), whose expression is upregulated as a consequence of terminal differentiation in human melanoma cells. Forced expression of MDA7 was found to be growth inhibitory toward diverse human tumor cells.

IL24 expression is up regulated in GPR30 KO mice as shown in FIG. 19.

### MMP12

### MMP12—Matrix Metalloproteinase 12

Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. It is thought that the protein encoded by this gene is cleaved at both ends to yield the active enzyme, but this processing has not been fully described. The enzyme degrades soluble and insoluble elastin. It may play a role in aneurysm formation and studies in mice suggest a role in the development of emphysema.

MMPs are involved in remodeling processes, fibrosis and tissues remodeling [Felkin et al. (2006)].

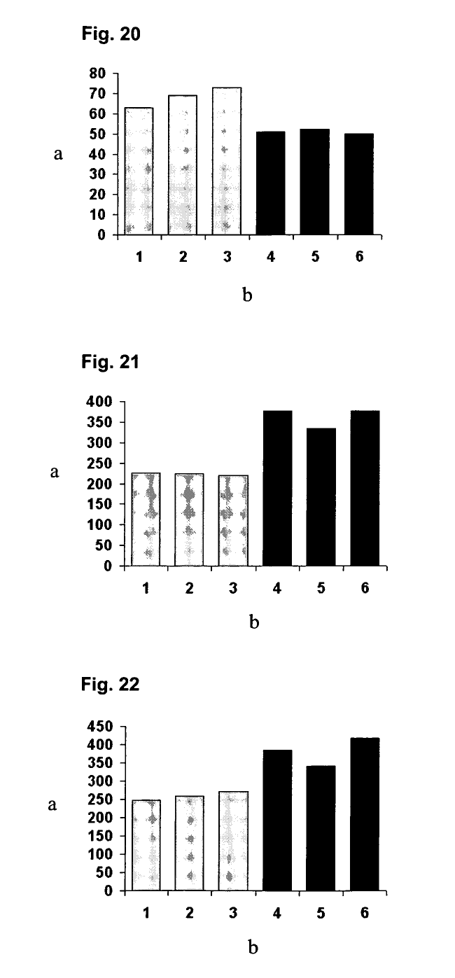

MMP12 expression is up regulated in GPR30 KO mice as shown in FIG. 20.

### BTG2

### BTG2—B-Cell Translocation Gene 2

BTG2 belongs to a family of structurally related proteins that appear to have antiproliferative properties. BTG2 is involved in the regulation of the G1/S transition of the cell cycle. Rouault [Rouault et al. (1996)] determined that BTG2 was preferentially expressed in quiescent cells and that overexpression of this gene causes a decrease in the growth rate and clonability of NIH 3T3 cells. Btg2 disruption had no detectable effect on the growth of differentiated or undifferentiated embryonic stem (ES) cells. Rouault et al. [Rouault et al. (1996)] reported that Btg2/Tis21 inactivation in ES cells leads to a striking disruption of DNA damage-induced G2/M arrest and to a marked increase in cell death. Rouault et al. [Rouault et al. (1996)] concluded that BTG2 function may be relevant to cell cycle control and to cellular response to DNA damage. They noted that in response to DNA damage, eukaryotic cells delay cell cycle progression from G1 to S and from G2 to M by induction of antiproliferative genes. Arrest in G1 is thought to prevent replication of damaged genetic templates; arrest prior to M allows cells to avoid segregation of defective chromosomes. Rouault et al. [Rouault et al. (1996)] determined that p53 (191170) regulates BTG2 gene expression.

BTG2 seems to be involved in myogenic differentiation [Feng et al. (2007)].

BTG2 expression is down regulated in GPR30 KO mice as shown in FIG. 21.

### Thbs

### Thbs—Thrombospondin 1

Thrombospondin I is a multimodular secreted protein that associates with the extracellular matrix and possesses a variety of biologic functions, including a potent antiangiogenic activity. Other thrombospondin genes include thrombospondins II (THBS2), III (THBS3), and IV (THBS4).

De Fraipont [De Fraipont et al. (2000)] measured the cytosolic concentrations of 3 proteins involved in angiogenesis, namely, platelet-derived endothelial cell growth factor (PDECGF), VEGFA, and THBS1 in a series of 43 human sporadic adrenocortical tumors. The tumors were classified as adenomas, transitional tumors, or carcinomas. PDECGF/thymidine phosphorylase levels were not significantly different among these 3 groups. One hundred percent of the adenomas and 73% of the transitional tumors showed VEGFA concentrations under the threshold value of 107 ng/g protein, whereas 75% of the carcinomas had VEGFA concentrations above this threshold value. Similarly, 89% of the adenomas showed THBS1 concentrations above the threshold value of 57 microg/g protein, whereas only 25% of the carcinomas and 33% of the transitional tumor samples did so. IGF2 overexpression, a common genetic alteration of adrenocortical carcinomas, was significantly correlated with higher VEGFA and lower THBS1 concentrations. The authors concluded that a decrease in THBS1 expression is an event that precedes an increase in VEGFA expression during adrenocortical tumor progression. The population of premalignant tumors with low THBS 1 and normal VEGFA levels could represent a selective target for antiangiogenic therapies.

Natural inhibitors of angiogenesis are able to block pathologic neovascularization without harming the preexisting vasculature. Volpert [Volpert et al. (2002)] concluded that this example of cooperation between pro- and antiangiogenic factors in the inhibition of angiogenesis provides one explanation for the ability of inhibitors to select remodeling capillaries for destruction.

Volpert [Volpert et al. (2002)] found that Idl is a potent inhibitor of Tspl transcription in mouse embryonic fibroblasts. In Idl null mice, upregulated expression of Tspl led to suppression of angiogenesis.

Using cDNA microarrays, Thakar [Thakar et al. (2005)] found that Tspl was the transcript showing highest induction at 3 hours following ischemia/reperfusion (IR) injury in rat and mouse kidneys. Northern blot analysis demonstrated that Tspl expression was undetectable at baseline, induced at 3 and 12 hours, and returned to baseline at 48 hours of reperfusion. Immunocytochemical staining showed injured proximal tubules were the predominant site of expression of Tsp1 in IR injury and that Tsp1 colocalized with activated caspase-3. Addition of purified Tspl to normal rat kidney proximal tubule cells or to cells subjected to ATP depletion in vitro induced injury, and knockout of Tsp1 in mice afforded significant protection against IR injury-induced renal failure and tubular damage. Thakar [Thakar et al. (2005)] concluded that TSP1 is a regulator of ischemic damage in the kidney and plays a role in the pathophysiology of ischemic renal failure.

Thbs1 seems to be involved in heart failure and remodeling processes [Vila et al. (2007)], [Vila et al. (2008)], [Belmadan et al. (2007)].

Thbs expression is down regulated in GPR30 KO mice as shown in FIG. 22.

### NR4a1

NR4a1—Nuclear Receptor Subfamily 4, Group A, Member 1 (NM_{—}173157, NM_{—}002135)

The Nerve Growth factor LB (NGFIB, also known as Nur77) protein is a member of the Nur nuclear receptor family[1] of intracellular transcription factors and is encoded by the NR4A1 gene (nuclear receptor subfamily 4, group A, member 1). NGFIB is involved in cell cycle mediation, inflammation and apoptosis. The NGFIB protein plays a key role in mediating inflammatory responses in macrophages. In addition, subcellular localization of the NGFIB protein appears to play a key role in the survival and death of cells.

Expression is induced by phytohemagglutinin in human lymphocytes and by serum stimulation of arrested fibroblasts. Translocation of the protein from the nucleus to mitochondria induces apoptosis. Multiple alternatively spliced variants, encoding the same protein, have been identified. Along with the two other Nur family members, NGFIB is expressed in macrophages following inflammatory stimuli. This process is mediated by the NF-κB (nuclear factor-kappa B) complex, a ubiquitous transcription factor involved in cellular response to stress.

NGFIB can be induced by many physiological and physical stimuli. These include physiological stimuli such as “fatty acids, stress, prostaglandins, growth factors, calcium, inflammatory cytokines, peptide hormones, phorbol esters, and neurotransmitters” and physical stimuli including “magnetic fields, mechanical agitation (causing fluid shear stress), and membrane depolarization”. Ligands do not bind to NGFIB, so modulation occurs at the level of protein expression and posttranslational modification.

NR4a1 expression is down regulated in GPR30 KO mice as shown in FIG. 23.

### ATF3

### Activating Transcription Factor 3; ATF3

An activating transcription factor (ATF)-binding site is a promoter element present in a wide variety of viral and cellular genes, including E1A-inducible adenoviral genes and cAMP-inducible cellular genes.

It was demonstrated that ATF3 is a member of the mammalian activation transcription factor/cAMP responsive element-binding (CREB) protein family of transcription factors, actually represses transcription from promoters with ATF sites. The truncated ATF3 variant, which does not bind DNA, stimulates transcription and antagonizes the action of ATF3.

ATF3 expression is regulated in GPR30 KO mice as shown in FIG. 25.

### ADAMTS1

A Disintegrin-Like and Metalloproteinase with Thrombospondin Type 1 Motif, 1; ADAMTS1

Thrombospondin-1 (THBS1 associates with the extracellular matrix and inhibits angiogenesis in vivo. In vitro, THBS 1 blocks capillary-like tube formation and endothelial cell proliferation. The antiangiogenic activity is mediated by a region that contains 3 type 1 (properdin or thrombospondin) repeats. Sequence analysis predicted that the 950-amino acid ADAMTS1 protein shares 52% amino acid identity with ADAMTS8 and 83% identity with mouse Adamts1. ADAMTS 1 is a secreted protein that has an N-terminal signal peptide, a zinc metalloprotease domain containing a zinc-binding site, and a cysteine-rich region containing 2 putative disintegrin loops. The C terminus of ADAMTS1 has 3 heparin-binding thrombospondin domains with 6 cys and 3 trp residues. Southern blot analysis showed that ADAMTS1 is a single-copy gene distinct from that encoding ADAMTS8. Northern blot analysis detected a 4.6-kb ADAMTS1 transcript in all tissues tested, with highest expression in adrenal, heart, and placenta, followed by skeletal muscle, thyroid, stomach, and liver. In fetal tissues, highest expression was detected in kidney. SDS-PAGE analysis demonstrated that ADAMTS1 is expressed as a 110-kD protein, an 85-kD protein after cleavage at the subtilisin site, or as a 67-kD protein, which is most abundant, generated by an additional processing event. Functional analysis determined that ADAMTS 1 disrupts angiogenesis in vivo and in vitro more efficiently than ADAMTS8, THBS1, or endostatin.

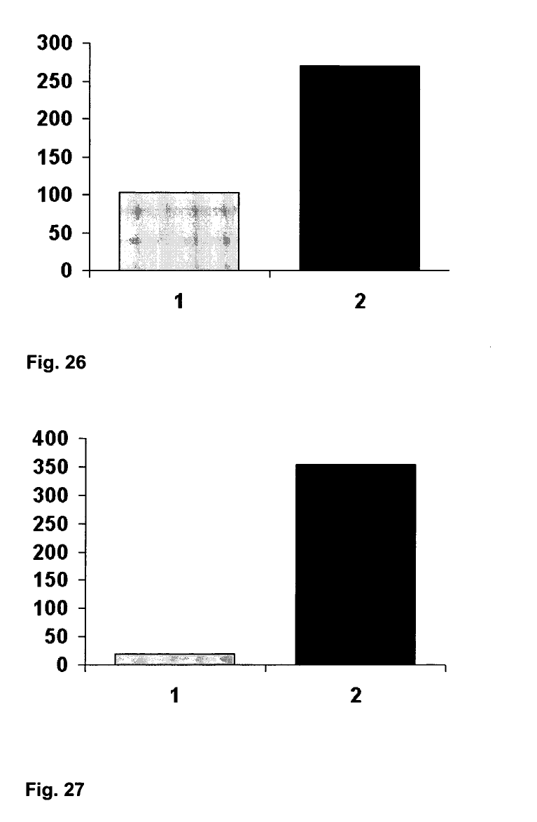

ADAMTS1 expression is regulated in GPR30 KO mice as shown in FIG. 26.

### KAP

kidney androgen regulated protein, KAP

KAP expression is regulated in GPR30 KO mice as shown in FIG. 27.

### SLC27a2

Solute Carrier Family 27 (Fatty Acid Transporter), Member 2; SLC27A2

In mammals, oxidation of very long chain fatty acids (VLCFAs) containing more than 22 carbons takes place primarily in peroxisomes. Very long chain acyl-CoA synthetase (VLACS), a peroxisomal and microsomal enzyme, catalyzes a crucial step in this pathway, the activation of VLCFAs to their CoA thioesters.

It was demonstrated that highest levels of Vies activity in mouse liver and kidney, tissues that showed highest expression of Vlcs by Northern blot and RT-PCR analyses. They used the mouse model of X-linked adrenoleukodystrophy (ALD) to test the hypothesis that the ALD protein (ABCD1) is required for proper expression or localization of VLCS. The results indicate that, although the beta-oxidation defect in mouse ALD fibroblasts improved with overexpression and targeting of Vlcs to peroxisomes, Ald protein was not necessary for the proper expression or localization of Vlcs, and the control of very long chain fatty acid levels did not depend on the direct interaction between Vlcs and Ald protein.

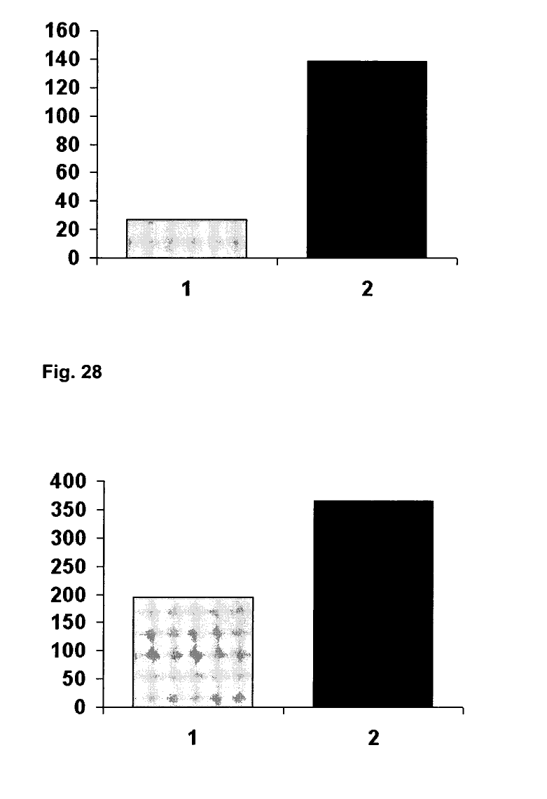

SLC27a2 expression is regulated in GPR30 KO mice as shown in FIG. 28.

### GPR30 in Cardiomyopathy

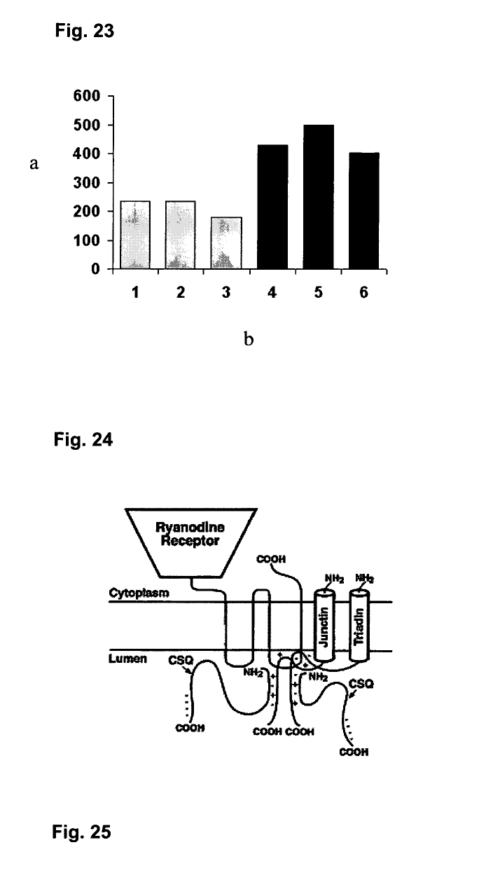

Ultrastructural and biochemical evidence suggests that a protein complex exists at the junctional SRI membrane in cardiac and skeletal muscle to facilitate the release of Calcium which occurs during muscle contraction. Components of this protein complex identified to date include the ryanodine receptor or Calcium release channel, which is visualized by electron microscopy as projecting feet on the cytoplasmic surface of the junctional membrane; calsequestrin, a high capacity Calcium-binding protein located in the junctional SR lumen, which buffers the calcium that is released during muscle contraction; and triadin and junctin, putative “anchoring” proteins, which appear to stabilize calsequestrin at the inner face of the junctional SR membrane. Calsequestrin is seen by electron microscopy as an electron-dense matrix in the SR lumen, where the protein appears to be physically connected to ryanodine receptors by “anchoring strands” or “rope-like fibers”. Biochemical evidence suggests that calsequestrin actively participates in muscle contraction by regulating the amount of Calcium released by the ryanodine receptor. This regulatory effect may be mediated by calsequestrin-anchoring proteins such as triadin and junction [Zang et al. (1997)]. The cartoon of the protein complex is shown in FIG. 24.

Junctin and triadin are integral membrane proteins sharing structural and amino acid sequence similarity which co-localize with the ryanodine receptor and calsequestrin at the junctional SR membrane in cardiac and skeletal muscle.

We have found that Trdn is upregulated in GPR30 knock out mice, which leads to a modulation of described Trdn protein complex. This complex is highly involved in the calcium pathway and signaling of the human heart. Alterations in intracellular Calcium homeostasis play a crucial role in heart failure, and abnormal cardiac ryanodine receptor function is recognized as a potential contributor of this disease. Uncontrolled ryanodine receptors gating is expected to result in increased diastolic SR Calcium leak causing a reduction of the SR calcium content, thus leading to reduced cardiac contractility. Altered composition of the ryanodine receptor channel complex due to altered expression of components of this complex, for example Trdn, may contribute to the altered ryanodine receptor function leading to cardiomyopathy [Gyorke et al. (2008)].

These data, together with the physiological characterization of the GPR30 knockout, demonstrate that GPR30 is a drug target for cardiovascular disease. Therefore, screening for GPR30 agonists/activators as potential therapeutic agents serves as basis for new therapeutic interventions treating cardiovascular diseases.

The data of the GPR30 knockout animals demonstrate that GPR30 deficient animals serve as animal models studying cardiovascular disease, especially cardiomyopathy. Such an animal model is suitable to assess the therapeutic capacity of potential therapeutic agents of various origine. Such agents can be agonists/antagonists of dall rug targets other than GPR30 itself.

Cells originated from such a GPR30 deficient animal or cell lines derived therefrom serve as a tool to study the mechanism of cardiovascular disease, especially the mechanism described above.

### Screening/Screening Assays

### Agonists of GPR30

Agonists as used herein, refer to compounds that activate GPR30 in vivo and/or in vivo. Agonists of GPR30 are molecules which, when bound to GPR30, increase or prolong the activity of GPR30. Agonists can be compounds that exert their effect on the GPR30 activity via the expression, via post-translational modifications or by other means. Agonists of GPR30 include proteins, nucleic acids, carbohydrates, small molecules, or any other molecule which activate GPR30.

The term “modulate”, as it appears herein, refers to a change in the activity of GPR30 polypeptide. For example, modulation may cause an increase or a decrease in protein activity, binding characteristics, or any other biological, functional, or immunological properties of GPR30.

As used herein, the terms “specific binding” or “specifically binding” refer to that interaction between a protein or peptide and an agonist or an antibody. The interaction is dependent upon the presence of a particular structure of the protein recognized by the binding molecule (i.e., the antigenic determinant or epitope). For example, if an antibody is specific for epitope “A” the presence of a polypeptide containing the epitope A, or the presence of free unlabeled A, in a reaction containing free labeled A and the antibody will reduce the amount of labeled A that binds to the antibody.

The invention provides methods (also referred to herein as “screening assays”) for identifying compounds which can be used for the treatment of cardiovascular diseases. The methods entail the identification of candidate or test compounds or agents (e.g., peptides, peptidomimetics, small molecules or other molecules) which bind to GPR30 and/or have a stimulatory effect on the biological activity of GPR30 or its expression and then determining which of these compounds have an effect on symptoms or diseases regarding the cardiovascular diseases in an in vivo assay.

Candidate or test compounds or agents which bind to GPR30 and/or have a stimulatory effect on the activity or the expression of GPR30 are identified either in assays that employ cells which express GPR30 on the cell surface (cell-based assays) or in assays with isolated GPR30 (cell-free assays). The various assays can employ a variety of variants of GPR30 (e.g., full-length GPR30, a biologically active fragment of GPR30, or a fusion protein which includes all or a portion of GPR30). Moreover, GPR30 can be derived from any suitable mammalian species (e.g., human GPR30, rat GPR30 or murine GPR30). The assay can be a binding assay entailing direct or indirect measurement of the binding of a test compound or a known GPR30 ligand to GPR30. The assay can also be an activity assay entailing direct or indirect measurement of the activity of GPR30. The assay can also be an expression assay entailing direct or indirect measurement of the expression of GPR30 mRNA or GPR30 protein. The various screening assays are combined with an in vivo assay entailing measuring the effect of the test compound on the symptoms of cardiovascular diseases.

In one embodiment, the invention provides assays for screening candidate or test compounds which bind to or modulate the activity of a membrane-bound (cell surface expressed) form of GPR30. Such assays can employ full-length GPR30, a biologically active fragment of GPR30, or a fusion protein which includes all or a portion of GPR30. As described in greater detail below, the test compound can be obtained by any suitable means, e.g., from conventional compound libraries. Determining the ability of the test compound to bind to a membrane-bound form of GPR30 can be accomplished, for example, by coupling the test compound with a radioisotope or enzymatic label such that binding of the test compound to the GPR30-expressing cell can be measured by detecting the labeled compound in a complex. For example, the test compound can be labelled with ¹²⁵I, ³⁵S, ¹⁴C, or ³H, either directly or indirectly, and the radioisotope detected by direct counting of radioemmission or by scintillation counting. Alternatively, the test compound can be enzymatically labelled with, for example, horseradish peroxidase, alkaline phosphatase, or luciferase, and the enzymatic label detected by determination of conversion of an appropriate substrate to product.

In a competitive binding format, the assay comprises contacting GPR30 expressing cell with a known compound which binds to GPR30 to form an assay mixture, contacting the assay mixture with a test compound, and determining the ability of the test compound to interact with the GPR30 expressing cell, wherein determining the ability of the test compound to interact with the GPR30 expressing cell comprises determining the ability of the test compound to preferentially bind the GPR30 expressing cell as compared to the known compound.

In another embodiment, the assay is a cell-based assay comprising contacting a cell expressing a membrane-bound form of GPR30 (e.g., full-length GPR30, a biologically active fragment of GPR30, or a fusion protein which includes all or a portion of GPR30) expressed on the cell surface with a test compound and determining the ability of the test compound to modulate (e.g., stimulate) the activity of the membrane-bound form of GPR30. Determining the ability of the test compound to modulate the activity of the membrane-bound form of GPR30 can be accomplished by any method suitable for measuring the activity of GPR30, e.g., any method suitable for measuring the activity of a G-protein coupled receptor or other seven-transmembrane receptor (described in greater detail below). The activity of a seven-transmembrane receptor can be measured in a number of ways, not all of which are suitable for any given receptor. Among the measures of activity are: alteration in intracellular Ca²⁺ concentration, activation of phospholipase C, alteration in intracellular inositol triphosphate (IP₃) concentration, alteration in intracellular diacylglycerol (DAG) concentration, and alteration in intracellular adenosine cyclic 3′, 5′-monophosphate (cAMP) concentration.

Determining the ability of the test compound to modulate the activity of GPR30 can be accomplished, for example, by determining the ability of GPR30 to bind to or interact with a target molecule. The target molecule can be a molecule with which GPR30 binds or interacts with in nature, for example, a molecule on the surface of a cell which expresses GPR30, a molecule on the surface of a second cell, a molecule in the extracellular milieu, a molecule associated with the internal surface of a cell membrane or a cytoplasmic molecule. The target molecule can be a component of a signal transduction pathway which facilitates transduction of an extracellular signal (e.g., a signal generated by binding of a GPR30 ligand, through the cell membrane and into the cell. The target GPR30 molecule can be, for example, a second intracellular protein which has catalytic activity or a protein which facilitates the association of downstream signaling molecules with GPR30.

Determining the ability of GPR30 to bind to or interact with a target molecule can be accomplished by one of the methods described above for determining direct binding. In one embodiment, determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca²⁺, diacylglycerol, IP₃, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response.

The present invention also includes cell-free assays. Such assays involve contacting a form of GPR30 (e.g., full-length GPR30, a biologically active fragment of GPR30, or a fusion protein comprising all or a portion of GPR30) with a test compound and determining the ability of the test compound to bind to GPR30. Binding of the test compound to GPR30 can be determined either directly or indirectly as described above. In one embodiment, the assay includes contacting GPR30 with a known compound which binds GPR30 to form an assay mixture, contacting the assay mixture with a test compound, and determining the ability of the test compound to interact with GPR30, wherein determining the ability of the test compound to interact with GPR30 comprises determining the ability of the test compound to preferentially bind to GPR30 as compared to the known compound.

The cell-free assays of the present invention are amenable to use of either a membrane-bound form of GPR30 or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include but are not limited to non-ionic detergents such as n-octylglucoside, n-dodecylglucoside, n-dodecylmaltoside, octanoyl-N-methylglucamide, decanoyl-N-methyl-glucamide, Triton X-100, Triton X-114, Thesit, Isotridecypoly(ethylene glycol ether)n, 3-[(3-cholamidopropyl) dimethylamminio]-1-propane sulfonate (CHAPS), 3-[(3-cholamidopropyl)di-methylamminio]-2-hydroxy-1-propane sulfonate (CHAPSO), or N-dodecyl=N,N-dimethyl-3-ammonio-1-propane sulfonate.

In various embodiments of the above assay methods of the present invention, it may be desirable to immobilize GPR30 (or a GPR30 target molecule) to facilitate separation of complexed from un-complexed forms of one or both of the proteins, as well as to accommodate automation of the assay. Binding of a test compound to GPR30, or interaction of GPR30 with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or GPR30, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of GPR30 can be determined using standard techniques.

Other techniques for immobilizing proteins on matrices can also be used in the screening assays of the invention. For example, either GPR30 or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated plates (Pierce Chemical). Alternatively, antibodies reactive with GPR30 or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with GPR30 or target molecule, as well as enzyme-linked assays which rely on detecting an enzymatic activity associated with GPR30 or target molecule.

The screening assay can also involve monitoring the expression of GPR30. For example, regulators of expression of GPR30 can be identified in a method in which a cell is contacted with a candidate compound and the expression of GPR30 protein or mRNA in the cell is determined. The level of expression of GPR30 protein or mRNA the presence of the candidate compound is compared to the level of expression of GPR30 protein or mRNA in the absence of the candidate compound. The candidate compound can then be identified as a regulator of expression of GPR30 based on this comparison. For example, when expression of GPR30 protein or mRNA protein is greater (statistically significantly greater) in the presence of the candidate compound than in its absence, the candidate compound is identified as a stimulator of GPR30 protein or mRNA expression. The level of GPR30 protein or mRNA expression in the cells can be determined by methods described below.

### Binding Assays

For binding assays, the test compound is preferably a small molecule which binds to and occupies the active site of GPR30 polypeptide, thereby making the ligand binding site inaccessible to substrate such that normal biological activity is prevented. Examples of such small molecules include, but are not limited to, small peptides or peptide-like molecules. Potential ligands which bind to a polypeptide of the invention include, but are not limited to, the natural ligands of known GPR30 GPCRs and analogues or derivatives thereof.

In binding assays, either the test compound or the GPR30 polypeptide can comprise a detectable label, such as a fluorescent, radioisotopic, chemiluminescent, or enzymatic label, such as horseradish peroxidase, alkaline phosphatase, or luciferase. Detection of a test compound which is bound to GPR30 polypeptide can then be accomplished, for example, by direct counting of radioemmission, by scintillation counting, or by determining conversion of an appropriate substrate to a detectable product. Alternatively, binding of a test compound to a GPR30 polypeptide can be determined without labeling either of the interactants. For example, a microphysiometer can be used to detect binding of a test compound with a GPR30 polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and GPR30 [Haseloff, (1988)].

Determining the ability of a test compound to bind to GPR30 also can be accomplished using a technology such as real-time Bimolecular Interaction Analysis (BIA) [McConnell, (1992); Sjolander, (1991)]. BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a GPR30-like polypeptide can be used as a “bait protein” in a two-hybrid assay or three-hybrid assay [Szabo, (1995); U.S. Pat. No. 5,283,317), to identify other proteins which bind to or interact with GPR30 and modulate its activity.

The two-hybrid system is based on the modular nature of most transcription factors, which consist of separable DNA-binding and activation domains. Briefly, the assay utilizes two different DNA constructs. For example, in one construct, polynucleotide encoding GPR30 can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with GPR30.

It may be desirable to immobilize either the GPR30 (or polynucleotide) or the test compound to facilitate separation of the bound form from unbound forms of one or both of the interactants, as well as to accommodate automation of the assay. Thus, either the GPR30-like polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach GPR30-like polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to GPR30 (or a polynucleotide encoding for GPR30) can be accomplished in any vessel suitable for containing the reactants. Examples of such vessels include microtiter plates, test tubes, and microcentrifuge tubes.

In one embodiment, GPR30 is a fusion protein comprising a domain that allows binding of GPR30 to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed GPR30; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined

Other techniques for immobilizing proteins or polynucleotides on a solid support also can be used in the screening assays of the invention. For example, either GPR30 (or a polynucleotide encoding GPR30) or a test compound can be immobilized utilizing conjugation of biotin and streptavidin. Biotinylated GPR30 (or a polynucleotide encoding biotinylated GPR30) or test compounds can be prepared from biotin-NHS (N-hydroxysuccinimide) using techniques well known in the art (e.g., biotinylation kit, Pierce Chemicals, Rockford, Ill.) and immobilized in the wells of streptavidin-coated plates (Pierce Chemical). Alternatively, antibodies which specifically bind to GPR30, polynucleotide, or a test compound, but which do not interfere with a desired binding site, such as the active site of GPR30, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to GPR30 polypeptide or test compound, enzyme-linked assays which rely on detecting an activity of GPR30 polypeptide, and SDS gel electrophoresis under non-reducing conditions.

Screening for test compounds which bind to a GPR30 polypeptide or polynucleotide also can be carried out in an intact cell. Any cell which comprises a GPR30 polypeptide or polynucleotide can be used in a cell-based assay system. A GPR30 polynucleotide can be naturally occurring in the cell or can be introduced using techniques such as those described above. Binding of the test compound to GPR30 or a polynucleotide encoding GPR30 is determined as described above.

### Functional Assays

Test compounds can be tested for the ability to increase GPR30 activity of a GPR30 polypeptide. The GPR30 activity can be measured, for example, using methods described in the specific examples, below. GPR30 activity can be measured after contacting either a purified GPR30, a cell membrane preparation, or an intact cell with a test compound. A test compound which increases GPR30 activity by at least about 10, preferably about 50, more preferably about 75, 90, or 100% is identified as a potential agent for increasing GPR30 activity.

One such screening procedure involves the use of melanophores which are transfected to express GPR30. Such a screening technique is described in PCT WO 92/01810 published Feb. 6, 1992. The screen may be employed for identifying a compound which activates the receptor by contacting such cells with compounds to be screened and determining whether each compound generates a signal, i.e., activates the receptor.

Other screening techniques include the use of cells which express GPR30 (for example, transfected CHO cells) in a system which measures extracellular pH changes caused by receptor activation [Iwabuchi, (1993)]. For example, compounds may be contacted with a cell which expresses the receptor polypeptide of the present invention and a second messenger response, e.g., signal transduction or pH changes, can be measured to determine whether the potential compound activates or inhibits the receptor. Another such screening technique involves introducing RNA encoding GPR30 into Xenopus oocytes to transiently express the receptor. The receptor oocytes can then be contacted with the receptor ligand and a compound to be screened, followed by detection of inhibition or activation of a calcium signal in the case of screening for compounds which are thought to inhibit activation of the receptor.

Another screening technique involves expressing GPR30 in cells in which the receptor is linked to a phospholipase C or D. Such cells include endothelial cells, smooth muscle cells, embryonic kidney cells, etc. The screening may be accomplished as described above by quantifying the degree of activation of the receptor from changes in the phospholipase activity.

### Gene Expression

In another embodiment, test compounds which increase or decrease GPR30 gene expression are identified. As used herein, the term “correlates with expression of a polynucleotide” indicates that the detection of the presence of nucleic acids, the same or related to a nucleic acid sequence encoding GPR30, by northern analysis or relatime PCR is indicative of the presence of nucleic acids encoding GPR30 in a sample, and thereby correlates with expression of the transcript from the polynucleotide encoding GPR30. The term “microarray”, as used herein, refers to an array of distinct polynucleotides or oligonucleotides arrayed on a substrate, such as paper, nylon or any other type of membrane, filter, chip, glass slide, or any other suitable solid support. A GPR30 polynucleotide is contacted with a test compound, and the expression of an RNA or polypeptide product of GPR30 polynucleotide is determined. The level of expression of appropriate mRNA or polypeptide in the presence of the test compound is compared to the level of expression of mRNA or polypeptide in the absence of the test compound. The test compound can then be identified as a regulator of expression based on this comparison. For example, when expression of mRNA or polypeptide is greater in the presence of the test compound than in its absence, the test compound is identified as a stimulator or enhancer of the mRNA or polypeptide expression.

The level of GPR30 mRNA or polypeptide expression in the cells can be determined by methods well known in the art for detecting mRNA or polypeptide. Either qualitative or quantitative methods can be used. The presence of polypeptide products of GPR30 polynucleotide can be determined, for example, using a variety of techniques known in the art, including immuno-chemical methods such as radioimmunoassay, Western blotting, and immunohistochemistry. Alternatively, polypeptide synthesis can be determined in vivo, in a cell culture, or in an in vitro translation system by detecting incorporation of labelled amino acids into GPR30.

Such screening can be carried out either in a cell-free assay system or in an intact cell. Any cell which expresses GPR30 polynucleotide can be used in a cell-based assay system. The GPR30 polynucleotide can be naturally occurring in the cell or can be introduced using techniques such as those described above. Either a primary culture or an established cell line can be used.

### Test Compounds

Suitable test compounds for use in the screening assays of the invention can be obtained from any suitable source, e.g., conventional compound libraries. The test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds [Lam, (1997)]. Examples of methods for the synthesis of molecular libraries can be found in the art. Libraries of compounds may be presented in solution or on beads, bacteria, spores, plasmids or phage.

### Modeling of Regulators

Computer modeling and searching technologies permit identification of compounds, or the improvement of already identified compounds, that can modulate GPR30 expression or activity. Having identified such a compound or composition, the active sites or regions are identified. Such active sites might typically be ligand binding sites, such as the interaction domain of the ligand with GPR30. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential GPR30 modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

### Therapeutic Indications and Methods

It was found by the present applicant that GPR30 is a diagnostic and therapeutic target for cardiovascular diseases, especially cardiomyopathy, hypertension, heart failure, myocardial infarction, coronary heart disease, myocardial ischemia, valvular diseases, atrial and ventricular arrhythmias, hypertensive vascular diseases, peripheral vascular diseases, stable and unstable angina pectoris, inflammatory cardiovascular diseases, pulmonary hypertension, shock, spasm of the coronary and peripheral arteries, thrombosis, thrombembolic disorders, stroke, edema, for example pulmonary or renal edema, restenosis, atherosclerosis and metabolic disorders.

The term “cardiomyopathy” according to the invention include, but is not limited to, cardiomyopathy (dilated, hypertrophic, restrictive, arrhythmogenic and unclassified cardiomyopathy), acute and chronic heart failure, right heart failure, left heart failure, biventricular heart failure, congenital heart defects, mitral valve stenosis, mitral valve insufficiency, aortic valve stenosis, aortic valve insufficiency, tricuspidal valve stenosis, tricuspidal valve insufficiency, pulmonal valve stenosis, pulmonal valve insufficiency, combined valve defects, myocarditis, acute myocarditis, chronic myocarditis, viral myocarditis, diastolic heart failure, systolic heart failure, diabetic heart failure and accumulation diseases.

Biomarker classes

GPR30 could be used as a biomarker for cardiovascular diseases in different classes:
Disease Biomarker: a biomarker that relates to a clinical outcome or measure of disease.
Efficacy Biomarker: a biomarker that reflects beneficial effect of a given treatment.
Staging Biomarker: a biomarker that distinguishes between different stages of a chronic disorder.
Surrogate Biomarker: a biomarker that is regarded as a valid substitute for a clinical outcomes measure.
Toxicity Biomarker: a biomarker that reports a toxicological effect of a drug on an in vitro or in vivo system.
Mechanism Biomarker: a biomarker that reports a downstream effect of a drug.
Target Biomarker: a biomarker that reports interaction of the drug with its target.

### Applications

The present invention provides GPR30 for prophylactic, therapeutic and diagnostic methods for cardiovascular diseases. The regulatory method of the invention involves contacting a cell with an agent that modulates one or more of the activities of GPR30. An agent that modulates activity can be an agent as described herein, such as a nucleic acid or a protein, a naturally-occurring cognate ligand of the polypeptide, a peptide, a peptidomimetic, or any small molecule. In one embodiment, the agent stimulates one or more of the biological activities of GPR30. Examples of such stimulatory agents include the active GPR30 and nucleic acid molecules encoding a portion of GPR30. In another embodiment, the agent inhibits one or more of the biological activities of GPR30. Examples of such inhibitory agents include antisense nucleic acid molecules and antibodies. These regulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by unwanted expression or activity of GPR30 or a protein in the GPR30 signaling pathway. In one embodiment, the method involves administering an agent like any agent identified or being identifiable by a screening assay as described herein, or combination of such agents that modulate say upregulate or downregulate the expression or activity of GPR30 or of any protein in the GPR30 signaling pathway. In another embodiment, the method involves administering a regulator of GPR30 as therapy to compensate for reduced or undesirably low expression or activity of GPR30 or a protein in the GPR30 signaling pathway.

Stimulation of activity or expression of GPR30 is desirable in situations in which activity or expression is abnormally low and in which increased activity is likely to have a beneficial effect. Conversely, inhibition of activity or expression of GPR30 is desirable in situations in which activity or expression of GPR30 is abnormally high and in which decreasing its activity is likely to have a beneficial effect.

This invention is further illustrated by the following examples which should not be construed as limiting. The contents of all references, patents and published patent applications cited throughout this application are hereby incorporated by reference.

### Pharmaceutical Compositions

This invention further pertains to novel agents identified by the above-described screening assays and uses thereof for treatments as described herein.

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes pharmaceutical compositions comprising a regulator of GPR30 expression or activity (and/or a regulator of the activity or expression of a protein in the GPR30 signaling pathway) as well as methods for preparing such compositions by combining one or more such regulators and a pharmaceutically acceptable carrier. Also within the invention are pharmaceutical compositions comprising a regulator identified using the screening assays of the invention packaged with instructions for use. For regulators that are agonists of GPR30 activity or increase GPR30 expression, the instructions would specify use of the pharmaceutical composition for treatment of cardiovascular diseases.

In another embodiment of the invention, the polynucleotides encoding GPR30, or any fragment or complement thereof, may be used for therapeutic purposes. In one aspect, the complement of the polynucleotide encoding GPR30 may be used in situations in which it would be desirable to block the transcription of the mRNA. In particular, cells may be transformed with sequences complementary to polynucleotides encoding GPR30. Thus, complementary molecules or fragments may be used to modulate GPR30 activity, or to achieve regulation of gene function. Such technology is now well known in the art, and sense or antisense oligonucleotides or larger fragments can be designed from various locations along the coding or control regions of sequences encoding GPR30.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors which will express nucleic acid sequence complementary to the polynucleotides of the gene encoding GPR30. These techniques are described, for example, in [Scott and Smith (1990) Science 249:386-390].

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical composition containing GPR30 in conjunction with a pharmaceutically acceptable carrier, for any of the therapeutic effects discussed above. Such pharmaceutical compositions may consist of GPR30, antibodies to GPR30, and mimetics or agonists of GPR30. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration. For pharmaceutical compositions which include an antagonist of GPR30 activity, a compound which reduces expression of GPR30, or a compound which reduces expression or activity of a protein in the GPR30 signaling pathway or any combination thereof, the instructions for administration will specify use of the composition for cardiovascular diseases,. For pharmaceutical compositions which include an agonist of GPR30 activity, a compound which increases expression of GPR30, or a compound which increases expression or activity of a protein in the GPR30 signaling pathway or any combination thereof, the instructions for administration will specify use of the composition for cardiovascular diseases.

### Diagnostics

In another embodiment, antibodies which specifically bind GPR30 may be used for the diagnosis of disorders characterized by the expression of GPR30, or in assays to monitor patients being treated with GPR30 or agonists, antagonists, and inhibitors of GPR30. Antibodies useful for diagnostic purposes may be prepared in the same manner as those described above for therapeutics. Diagnostic assays for GPR30 include methods which utilize the antibody and a label to detect GPR30 in human body fluids or in extracts of cells or tissues. The antibodies may be used with or without modification, and may be labeled by covalent or non-covalent joining with a reporter molecule. A wide variety of reporter molecules, several of which are described above, are known in the art and may be used.

A variety of protocols for measuring GPR30, including ELISAs, RIAs, and FACS, are known in the art and provide a basis for diagnosing altered or abnormal levels of GPR30 expression. Normal or standard values for GPR30 expression are established by combining body fluids or cell extracts taken from normal mammalian subjects, preferably human, with antibody to GPR30 under conditions suitable for complex formation The amount of standard complex formation may be quantified by various methods, preferably by photometric means. Quantities of GPR30 expressed in subject samples from biopsied tissues are compared with the standard values. Deviation between standard and subject values establishes the parameters for diagnosing disease.

In another embodiment of the invention, the polynucleotides encoding GPR30 may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of GPR30 may be correlated with disease. The diagnostic assay may be used to distinguish between absence, presence, and excess expression of GPR30, and to monitor regulation of GPR30 levels during therapeutic intervention.

Polynucleotide sequences encoding GPR30 may be used for the diagnosis of cardiovascular diseases. The polynucleotide sequences encoding GPR30 may be used in Southern, Northern, or dot-blot analysis, or other membrane-based technologies; in PCR technologies; in dipstick, pin, and ELISA assays; and in microarrays utilizing fluids or tissues from patient biopsies to detect altered GPR30 expression. Such qualitative or quantitative methods are well known in the art.

In a particular aspect, the nucleotide sequences encoding GPR30 may be useful in assays that detect the presence of associated disorders, particularly those mentioned above. The nucleotide sequences encoding GPR30 may be labelled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered from that of a comparable control sample, the nucleotide sequences have hybridized with nucleotide sequences in the sample, and the presence of altered levels of nucleotide sequences encoding GPR30 in the sample indicates the presence of the associated disorder. Such assays may also be used to evaluate the efficacy of a particular therapeutic treatment regimen in animal studies, in clinical trials, or in monitoring the treatment of an individual patient.

In order to provide a basis for the diagnosis of cardiovascular diseases associated with the expression of GPR30, a normal or standard profile for expression is established. This may be accomplished by combining body fluids or cell extracts taken from normal subjects, either animal or human, with a sequence, or a fragment thereof, encoding GPR30, under conditions suitable for hybridization or amplification. Standard hybridization may be quantified by comparing the values obtained from normal subjects with values from an experiment in which a known amount of a substantially purified polynucleotide is used. Standard values obtained from normal samples may be compared with values obtained from samples from patients who are symptomatic for a disorder. Deviation from standard values is used to establish the presence of a disorder.

Another technique for drug screening which may be used provides for high throughput screening of compounds having suitable binding affinity to the protein of interest as described in published PCT application WO84/03564. In this method, large numbers of different small test compounds are synthesized on a solid substrate, such as plastic pins or some other surface. The test compounds are reacted with GPR30, or fragments thereof, and washed. Bound GPR30 is then detected by methods well known in the art. Purified GPR30 can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding GPR30 specifically compete with a testcompound for binding GPR30. In this manner, antibodies can be used to detect the presence of any peptide which shares one or more antigenic determinants with GPR30.

### Determination of a Therapeutically Effective Dose

The determination of a therapeutically effective dose is well within the capability of those skilled in the art. A therapeutically effective dose refers to that amount of active ingredient which increases GPR30 activity relative to GPR30 activity which occurs in the absence of the therapeutically effective dose. For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀. Pharmaceutical compositions which exhibit large therapeutic indices are preferred. The data obtained from cell culture assays and animal studies are used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 micrograms to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun”, and DEAE- or calcium phosphate-mediated transfection.

If the expression product is mRNA, the reagent is preferably an antisense oligonucleotide or a ribozyme. Polynucleotides which express antisense oligonucleotides or ribozymes can be introduced into cells by a variety of methods, as described above. Preferably, a reagent reduces expression of GPR30 gene or the activity of GPR30 by at least about 10, preferably about 50, more preferably about 75, 90, or 100% relative to the absence of the reagent. The effectiveness of the mechanism chosen to decrease the level of expression of GPR30 gene or the activity of GPR30 can be assessed using methods well known in the art, such as hybridization of nucleotide probes to GPR30-specific mRNA, quantitative RT-PCR, immunologic detection of GPR30, or measurement of GPR30 activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects. Any of the therapeutic methods described above can be applied to any subject in need of such therapy, including, for example, mammals such as dogs, cats, cows, horses, rabbits, monkeys, and most preferably, humans.

An object of the invention is a transgenic non-human animal which is deficient in expressing GPR30 polypeptide due to a deficient GPR30 gene.

An object of the invention is a transgenic non-human animal (“animal model”) model which is deficient in expressing GPR30 polypeptide due to a deficient GPR30 gene characterized in that it displays cardiovascular defects.

In another embodiment deficient in expressing GPR30 polypeptide comprises deficiencies due to not expressing functional GPR30 polypeptide or expressing suboptimal levels of GPR30 polypeptide.

Another object of the invention is a transgenic non-human animal or animal model exhibiting cardiovascular defects wherein said animal does not express functional GPR30 polypeptide or expresses suboptimal levels of GPR30 polypeptide. Suboptimal expression levels of GPR30 are levels which lead to a cardiovascular defect, a cardiomyopathy or to defects characterized by an increase in LVEDP and tau and a decrease in LVdPdtₘₐₓ and LVdPdtₘᵢₙ.

An object of the invention is a transgenic non-human animal or animal model exhibiting cardiovascular defects characterized in that said transgenic non-human animal displays an increase in LVEDP and tau and a decrease in LVdPdtₘₐₓ and LVdPdtₘᵢₙ compared to wild type wherein said transgenic non-human does not express functional GPR30 polypeptide or express suboptimal levels of GPR30 polypeptide.

In one embodiment the said transgenic non-human animal is a mammal, more preferably a rodent animal, preferably mouse, rabbit, hamster or guinea pig, most preferred is a mouse. In another embodiment said animal does not express functional GPR30 polypeptide due to homozygous disruption of the GPR30 gene. In another embodiment said animal does express suboptimal levels of GPR30 polypeptide due to heterozygous disruption of the GPR30 gene, due to RNAi- or siRNA-mediated silencing of GPR30 expression or due to mutations inserted in the GPR30 gene reducing the activity of the GPR30 activity. In yet another embodiment of the invention cardiovascular defects are defects comprised in a group of defects consisting of cardiomyopathy, hypertension, heart failure, myocardial infarction, coronary heart disease, myocardial ischemia, valvular diseases, atrial and ventricular arrhythmias, hypertensive vascular diseases, peripheral vascular diseases, stable and unstable angina pectoris, inflammatory cardiovascular diseases, pulmonary hypertension, shock, spasm of the coronary and peripheral arteries, thrombosis, thrombembolic disorders, stroke, edema, for example pulmonary or renal edema, restenosis and metabolic disorders. In a preferred embodiment a cardiovascular defect is a cardiomyopathy.

An object of the invention is a transgenic non-human animal model exhibiting a cardiomyopathy wherein said animal does not expresses functional GPR30 polypeptide or express suboptimal levels of GPR30 polypeptide.

An object of the invention is a transgenic non-human animal model exhibiting a cardiomyopathy characterized in that said transgenic non-human animal displays an increase in LVEDP and tau and a decrease in LVdPdtₘₐₓ and LVdPdtₘᵢₙ compared to wild type wherein said transgenic non-human animal does not express functional GPR30 polypeptide or express suboptimal levels of GPR30 polypeptide.

An object of the invention is a transgenic non-human animal model characterized in that said transgenic non-human animal displays cardiac parameters with an increase in LVEDP and tau and a decrease in LVdPdtₘₐₓ and LVdPdtₘᵢₙ compared to wild type wherein said transgenic non-human animal does not express functional GPR30 polypeptide or express suboptimal levels of GPR30 polypeptide.

An object of the invention is a method for selecting a potential therapeutic agent for treating a cardiovascular defect occurring in the animal model described above comprising administering one ore more agents to be tested to the animal model of above and determining whether said cardiovascular defect occurring in said animal model have changed as a result of administration of said agent or agents. A preferred embodiment of the above method is the determination whether said cardiovascular defect is ameliorated.

Another object of the invention is a method for selecting a potential therapeutic agent for treating a cardiomyopathy in the animal model described above comprising administering one ore more agents to be tested to the animal model of above and determining whether said cardiomyopathy occurring in said animal model have changed as a result of administration of said agent or agents wherein said determination comprises the measurement of LVEDP, tau LVdPdtₘₐₓ and/or LVdPdtₘᵢₙ. A preferred embodiment of the above method is the determination whether said cardiomyopathy is ameliorated.

Another object of the invention is a method for the screening of drug candidates, characterized in that the anti-cardiomyopathic effect of said drug candidates is assessed in a non-human transgenic animal according to the animal model described above or a cell thereof or a cell line derived therefrom.

In another embodiment the above methods are with the provision that methods of treatment of the animal body by surgery or therapy and diagnostic methods practiced on the animal body are excluded.

An object of the invention is a method of screening for therapeutic agents useful in the treatment of cardiovascular diseases in a mammal comprising the steps of (i) contacting a test compound with a GPR30 polypeptide, (ii) detect binding of said test compound to said GPR30 polypeptide. E.g., compounds that bind to the GPR30 polypeptide are identified potential therapeutic agents for such a disease, wherein an agent is selected as useful in the treatment of the aforementioned defects if it increases the activity of a GPR30 polypeptide.

An object of the invention is a method of screening for therapeutic agents useful in the treatment of a cardiac defect characterized by an increase in LVEDP and tau and a decrease in LVdPdtₘₐₓ and LVdPdtₘᵢₙ in a mammal comprising the steps of (i) contacting a test compound with a GPR30 polypeptide, (ii) detect binding of said test compound to said GPR30 polypeptide. E.g., compounds that bind to the GPR30 polypeptide are identified potential therapeutic agents for the aforementioned cardiac defect, wherein an agent is selected as useful in the treatment of the aforementioned defects if it increases the activity of a GPR30 polypeptide.

An object of the invention is a method of screening for therapeutic agents useful in the treatment of a cardiac defect characterized by an increase in LVEDP and/or tau and/or a decrease in LVdPdtₘₐₓ and/or LVdPdtₘᵢₙ in a mammal comprising the steps of (i) contacting a test compound with a GPR30 polypeptide, (ii) detect binding of said test compound to said GPR30 polypeptide. E.g., compounds that bind to the GPR30 polypeptide are identified potential therapeutic agents for the aforementioned cardiac defect, wherein an agent is selected as useful in the treatment of the aforementioned defects if it increases the activity of a GPR30 polypeptide.

Another object of the invention is a method of screening for therapeutic agents useful in the treatment of cardiac defect characterized by an increase in LVEDP and tau and a decrease in LVdPdtₘₐₓ and LVdPdtₘᵢₙ wherein in a mammal comprising the steps of (i) contacting a test compound with a GPR30 polypeptide, and (ii) detect binding of said test compound to said GPR30 polypeptide, wherein an agent is selected as useful in the treatment of the aforementioned defects if it increases the activity of a GPR30 polypeptide.

Another object of the invention is a method of screening for therapeutic agents useful in the treatment of cardiovascular diseases in a mammal comprising the steps of (i) determining the activity of a GPR30 polypeptide at a certain concentration of a test compound or in the absence of said test compound, (ii) determining the activity of said polypeptide at a different concentration of said test compound. E.g., compounds that lead to a difference in the activity of the GPR30 polypeptide in (i) and (ii) are identified potential therapeutic agents for such a disease.

Another object of the invention is a method of screening for therapeutic agents useful in the treatment of a cardiac defect characterized by an increase in LVEDP and tau and a decrease in LVdPdtₘₐₓ and LVdPdtₘᵢₙ in a mammal comprising the steps of (i) determining the activity of a GPR30 polypeptide at a certain concentration of a test compound or in the absence of said test compound, (ii) determining the activity of said polypeptide at a different concentration of said test compound. E.g., compounds that lead to a difference in the activity of the GPR30 polypeptide in (i) and (ii) are identified potential therapeutic agents for the aforementioned cardiac defect, wherein an agent is selected as useful in the treatment of the aforementioned defects if it increases the activity of a GPR30 polypeptide.

Another object of the invention is a method of screening for therapeutic agents useful in the treatment of a cardiac defect characterized by an increase in LVEDP and/or tau and/or a decrease in LVdPdtₘₐₓ and/or LVdPdtₘᵢₙ in a mammal comprising the steps of (i) determining the activity of a GPR30 polypeptide at a certain concentration of a test compound or in the absence of said test compound, (ii) determining the activity of said polypeptide at a different concentration of said test compound. E.g., compounds that lead to a difference in the activity of the GPR30 polypeptide in (i) and (ii) are identified potential therapeutic agents for the aforementioned cardiac defect, wherein an agent is selected as useful in the treatment of the aforementioned defects if it increases the activity of a GPR30 polypeptide.

Another object of the invention is a method of screening for therapeutic agents useful in the treatment of cardiovascular diseases in a mammal comprising the steps of (i) determining the activity of a GPR30 polypeptide at a certain concentration of a test compound, (ii) determining the activity of a GPR30 polypeptide at the presence of a compound known to be a regulator of a GPR30 polypeptide. E.g., compounds that show similar effects on the activity of the GPR30 polypeptide in (i) as compared to compounds used in (ii) are identified potential therapeutic agents for such a disease.

Another object of the invention is a method of screening for therapeutic agents useful in the treatment of a cardiac defect characterized by an increase in LVEDP and tau and a decrease in LVdPdtₘₐₓ and LVdPdtₘᵢₙ in a mammal comprising the steps of (i) determining the activity of a GPR30 polypeptide at a certain concentration of a test compound, (ii) determining the activity of a GPR30 polypeptide at the presence of a compound known to be a regulator of a GPR30 polypeptide. E.g., compounds that show similar effects on the activity of the GPR30 polypeptide in (i) as compared to compounds used in (ii) are identified potential therapeutic agents for the aforementioned cardiac defect, wherein an agent is selected as useful in the treatment of the aforementioned defects if it increases the activity of a GPR30 polypeptide.

Another object of the invention is a method of screening for therapeutic agents useful in the treatment of a cardiac defect characterized by an increase in LVEDP and/or tau and/or a decrease in LVdPdtₘₐₓ and/or LVdPdtₘᵢₙ in a mammal comprising the steps of (i) determining the activity of a GPR30 polypeptide at a certain concentration of a test compound, (ii) determining the activity of a GPR30 polypeptide at the presence of a compound known to be a regulator of a GPR30 polypeptide. E.g., compounds that show similar effects on the activity of the GPR30 polypeptide in (i) as compared to compounds used in (ii) are identified potential therapeutic agents for the aforementioned cardiac defect, wherein an agent is selected as useful in the treatment of the aforementioned defects if it increases the activity of a GPR30 polypeptide.

Other objects of the invention are methods of the above, wherein the step of contacting is in or at the surface of a cell.

Other objects of the invention are methods of the above, wherein the cell is in vitro.

Other objects of the invention are methods of the above, wherein the step of contacting is in a cell-free system.

Other objects of the invention are methods of the above, wherein the polypeptide is coupled to a detectable label.

Other objects of the invention are methods of the above, wherein the compound is coupled to a detectable label.

Other objects of the invention are methods of the above, wherein the test compound displaces a ligand which is first bound to the polypeptide.

Other objects of the invention are methods of the above, wherein the polypeptide is attached to a solid support.

Other objects of the invention are methods of the above, wherein the compound is attached to a solid support.

Another object of the invention is a method of screening for therapeutic agents useful in the treatment of a disease comprised in a group of diseases consisting of cardiovascular diseases in a mammal comprising the steps of (i) contacting a test compound with a GPR30 polynucleotide, (ii) detect binding of said test compound to said GPR30 polynucleotide. Compounds that, e.g., bind to the GPR30 polynucleotide are potential therapeutic agents for the treatment of such diseases.

Another object of the invention is a method of screening for therapeutic agents useful in the treatment of a cardiac defect characterized by an increase in LVEDP and tau and a decrease in LVdPdtₘₐₓ and LVdPdtₘᵢₙ in a mammal comprising the steps of (i) contacting a test compound with a GPR30 polynucleotide, (ii) detect binding of said test compound to said GPR30 polynucleotide. Compounds that, e.g., bind to the GPR30 polynucleotide are potential therapeutic agents for the treatment of the aforementioned cardiac defect, wherein an agent is selected as useful in the treatment of the aforementioned defects if it increases the activity of a GPR30 polypeptide.

Another object of the invention is a method of screening for therapeutic agents useful in the treatment of a cardiac defect characterized by an increase in LVEDP and/or tau and/or a decrease in LVdPdtₘₐₓ and/or LVdPdtₘᵢₙ in a mammal comprising the steps of (i) contacting a test compound with a GPR30 polynucleotide, (ii) detect binding of said test compound to said GPR30 polynucleotide. Compounds that, e.g., bind to the GPR30 polynucleotide are potential therapeutic agents for the treatment of the aforementioned cardiac defect, wherein an agent is selected as useful in the treatment of the aforementioned defects if it increases the activity of a GPR30 polypeptide.

Another object of the invention is the method of the above, wherein the nucleic acid molecule is RNA.

Another object of the invention is a method of the above, wherein the contacting step is in or at the surface of a cell.

Another object of the invention is a method of the above, wherein the contacting step is in a cell-free system.

Another object of the invention is a method of the above, wherein the polynucleotide is coupled to a detectable label.

Another object of the invention is a method of the above, wherein the test compound is coupled to a detectable label.

Another object of the invention is a method of diagnosing cardiovascular diseases in a mammal comprising the steps of (i) determining the amount of a GPR30 polynucleotide in a sample taken from said mammal, (ii) determining the amount of GPR30 polynucleotide in healthy and/or diseased mammal. A disease is diagnosed, e.g., if there is a substantial similarity in the amount of GPR30 polynucleotide in said test mammal as compared to a diseased mammal.

Another object of the invention is a pharmaceutical composition for the treatment of cardiovascular diseases in a mammal comprising a therapeutic agent which binds to a GPR30 polypeptide.

Another object of the invention is a pharmaceutical composition for the treatment of cardiovascular diseases in a mammal comprising a therapeutic agent which stimulates the activity of a GPR30 polypeptide.

Another object of the invention is a pharmaceutical composition for the treatment of cardiovascular diseases in a mammal comprising a therapeutic agent which stimulates the activity of a GPR30 polypeptide, wherein said therapeutic agent is (i) a small molecule, (ii) an RNA molecule, (iii) an antisense oligonucleotide, (iv) a polypeptide, (v) an antibody, or (vi) a ribozyme.

Another object of the invention is a pharmaceutical composition for the treatment of cardiovascular diseases in a mammal comprising a GPR30 polynucleotide.

Another object of the invention is a pharmaceutical composition for the treatment of cardiovascular diseases in a mammal comprising a GPR30 polypeptide.

Another object of the invention is the use of regulators of a GPR30 for the preparation of a pharmaceutical composition for the treatment of cardiovascular diseases in a mammal.

Another object of the invention is a method for the preparation of a pharmaceutical composition useful for the treatment of cardiovascular diseases in a mammal comprising the steps of (i) identifying an agonist of GPR30, (ii) determining whether said agonist ameliorates the symptoms of cardiovascular diseases in a mammal; and (iii) combining of said agonists with an acceptable pharmaceutical carrier.

Another object of the invention is the use of an agonist of GPR30 for the stimulation of GPR30 activity in a mammal having a cardiovascular disease. The uses, methods or compositions of the invention are useful for cardiovascular diseases.

The hemodynamic measurements of the GPR30 knockout mice suggests a particular—but not limited to—utilization of GPR30 for diagnosis, use as a screening target and treatment of cardiovascular defects, preferrably cardiomyopathy.

The examples below are provided to illustrate the subject invention. These examples are provided by way of illustration and are not included for the purpose of limiting the invention.

### EXAMPLES

Hereinafter the present invention is explained in more detail with referring to the following examples, but the present invention is not limited thereto.

### Example 1

### Generation of GPR30 Knockout Mice

To inactivate Gpr30 in vivo, exon 3 encoding the complete open reading frame of Gpr30 was deleted from the murine genome. The targeting construct was based on a 9.8 kB genomic fragment representing the genomic sequence of exon 1, 2, and 3, and the surrounding introns of the mouse Gpr30 gene. This fragment, obtained from a RP23 BAC library, was modified using homologous recombination in E. coli to carry a loxP site 5′ to exon 3, a PGKtkneo cassette flanked by frt sites, and one loxP site in the 3′ direction of exon 3. C57BL/6N embryonic stem cells were transfected with the linearized targeting construct. After transfection of the embryonic stem cells, G418-resistant clones were analyzed by Southern blot using probes from outside the homology arms of the targeting vector. The 5′-external probe A detected a 10 kB KpnI-fragment from the endogenous allele in all clones, and an additional 12 kB KpnI-fragment in one allele that underwent homologous recombination with the targeting vector. The 3′-external probe B detected a 16.9 kB NheI-fragment from the wildtype allele in all clones, and an additional 5.7 kB NheI-fragment in all clones that were homologously recombined. Single integration of the targeting vector was analyzed by hybridization of the Southern blots with an internal probe derived from the PGKtkneo cassette. The frequency of homologous recombination was 3.2%. Two homologously recombined clones harboring the targeted allele were used for the generation of chimeric mice by blastocyst injection. Highly chimeric mice were bred to C57BL/6 females and offspring heterozygous for the targeted allele was identified by Southern blot. To eliminate the selection marker and exon 3, mice heterozygous for the targeted allele were bred with mice carrying one copy of the Cre recombinase transgene in their ROSA26 locus. The resulting offspring, heterozygous for the null allele was backcrossed with C57BL/6 mice to eliminate the Cre recombinase transgene. Wildtype and mutant experimental animals were derived from heterozygous intercrosses and were devoid of the Cre recombinase transgene.

### Example 2

### Analysis of Left Ventricular Hemodynamics of GPR30 Knockout and Wildtype Mice

The GPR30 knockout mouse of the present invention is a conventional knockout mouse with a C57BL/6 background.

Male GPR30 knockout mice and wildtype mice (16-20 weeks old, n=8-11 per group) were anethetized with isoflurane (1.8% vol/vol).

Core body temperature was maintained at 37° C. using a controlled heating pad.

A Millar microtip catheter (SPR-671, FMI Fohr Medical Instruments GmbH, Seeheim/Ober-Beerbach, Germany) was inserted through the right carotid artery into the left ventricle for measurement of left ventricular hemodynamics (left ventricular systolic pressure, left ventricular enddiastolic pressure, LVdPdtₘₐₓ, LVdPdtₘᵢₙ, tau). All hemodynamic measurements were performed with a PowerLab System using the Chart 5.0 Software (ADlnstruments GmbH, Spechbach, Germany).

LVdPdtₘₐₓ and LVdPdtₘᵢₙ of the GPR30 knockout mice were 8931±445 mmHg/s and −9423±367 mmHg/s respectively, which were significantly low as compared with those of the wildtype mice (LVdPdtₘₐₓ: 10406±400; LVdPdtₘᵢₙ: −12047±944) (FIGS. 3 and 6).

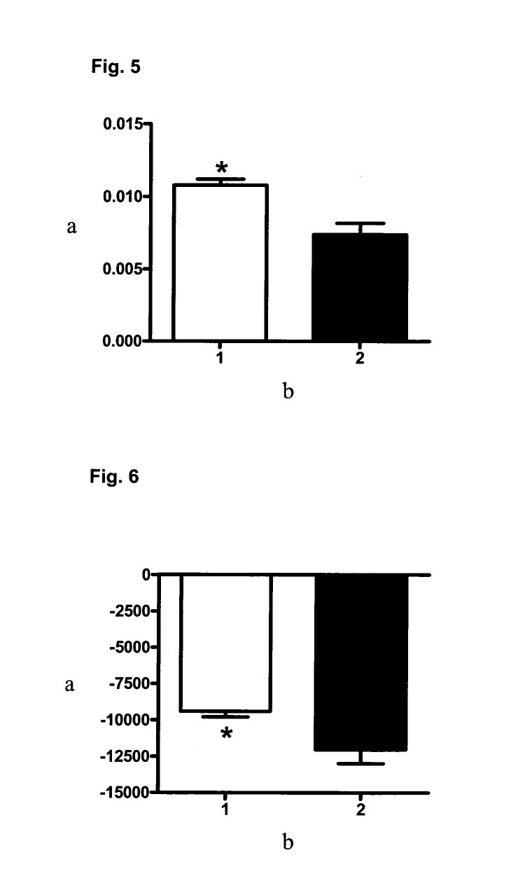

Left ventricular enddiastolic pressure (LVEDP) and the left ventricular relaxation time constant (tau) of the GPR30 knockout mice was increased by 7.3±0.6 mmHg and 0.011±0.0004 s respectively, which was significantly high as compared with those of the wildtype mice (LVEDP: 5.1±0.7; tau: 0.007±0.0008 s) (FIGS. 4 and 5).

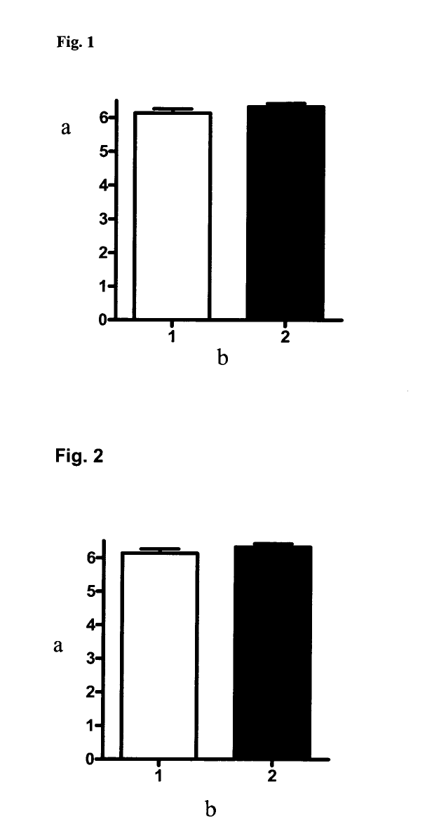

The left ventricular systolic pressure of the knockout mice did not show significant changes as compared with that of the wildtype mice (FIG. 2).

### Example 3

### Analysis of Heart Weight of GPR30 Knockout and Wildtype Mice

The relation of the heart weight to the tibia length for the GPR30 knockout was 6.1±0.1 mg/mm, whereas that of the wildtype mice was 6.3±0.1 mg/mm (FIG. 1).

### Example 4

### Analysis of ECG of GPR30 Knockout and Wildtype Mice

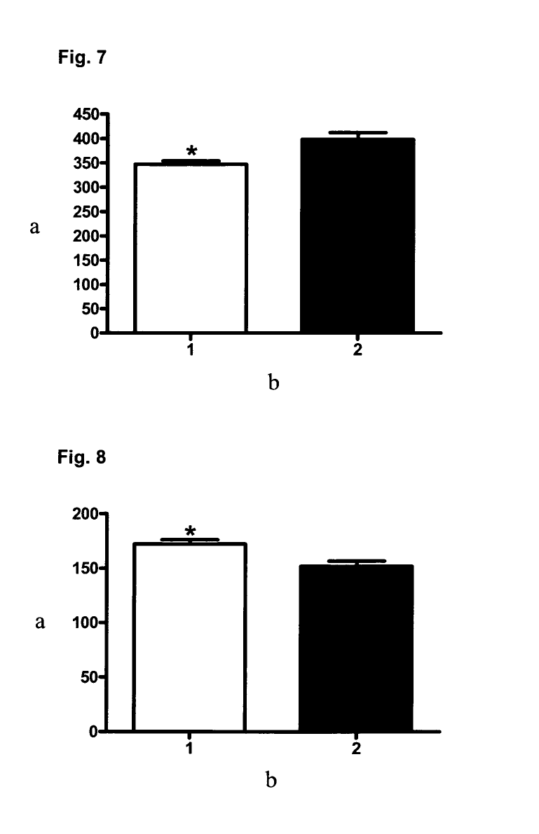

Male GPR30 knockout mice and wildtype mice (16-20 weeks old, n=10 per group) were anethetized with isoflurane (0.8% vol/vol) (FIGS. 7 and 8).

Core body temperature was maintained at 37° C. using a controlled heating pad.

ECG was performed with a PowerLab System using the Chart 5.0 Software (ADInstruments GmbH, Spechbach, Germany).

The heart rate of the GPR30 knockout mice was 348±7 beats per minute, which was significantly low as compared with that of the wildtype mice (398±14 bpm).

The RR interval of the GPR30 knockout mice was 172.3 ±3.9 ms, which was significantly elongated as compared with that of the wildtype mice (151.8 ±4.8 ms).

The PR interval of the knockout mice (39.0±0.7 ms) did not show significant changes as compared with that of the wildtype mice (38.5±0.4).

### Example 5

### Analysis of Exercise Capacity of GPR30 Knockout and Wildtype Mice

Exercise capacity of GPR30 knockout and wildtype mice (16-20 weeks old, n=5 per group) was investigated by voluntary wheel running. The mice were single housed. A running wheel was placed in each cage, without any other form of environmental enrichment. Wheel running activity was monitored from 4 p.m. to 8 a.m. using a light barrier. After one week running-in period, the running activity was recorded for three weeks.

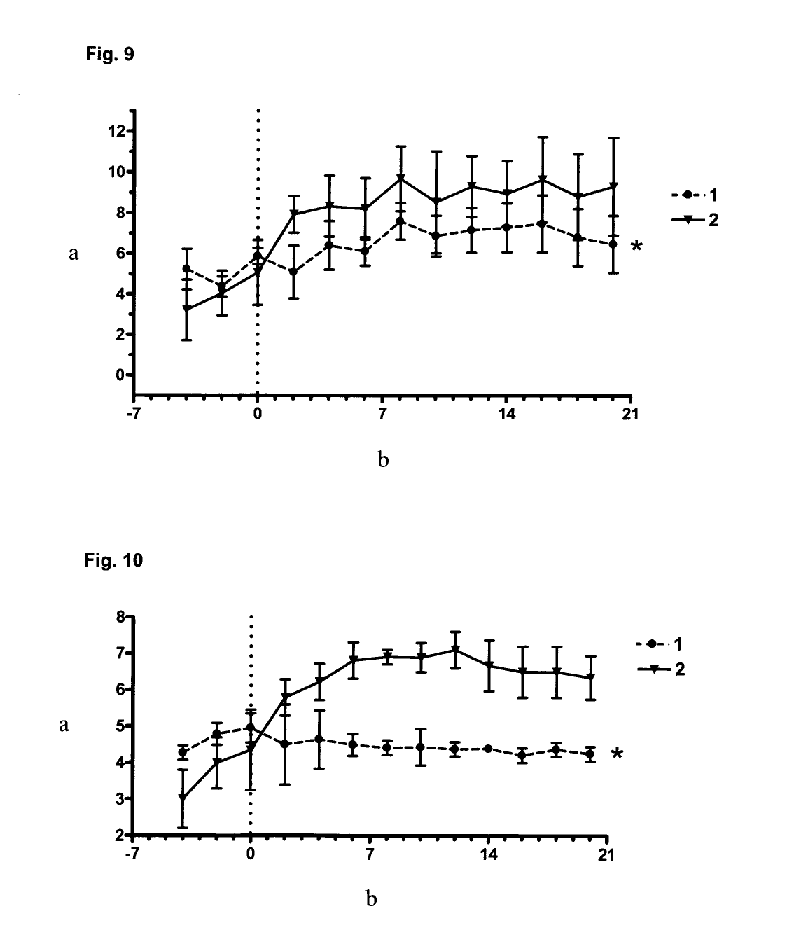

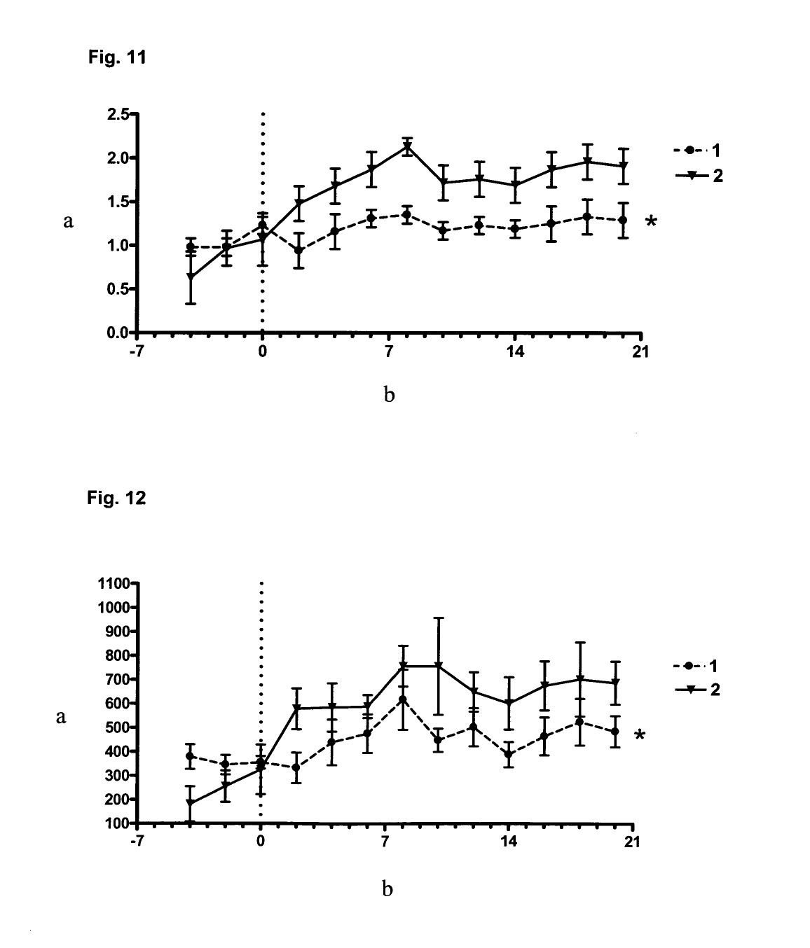

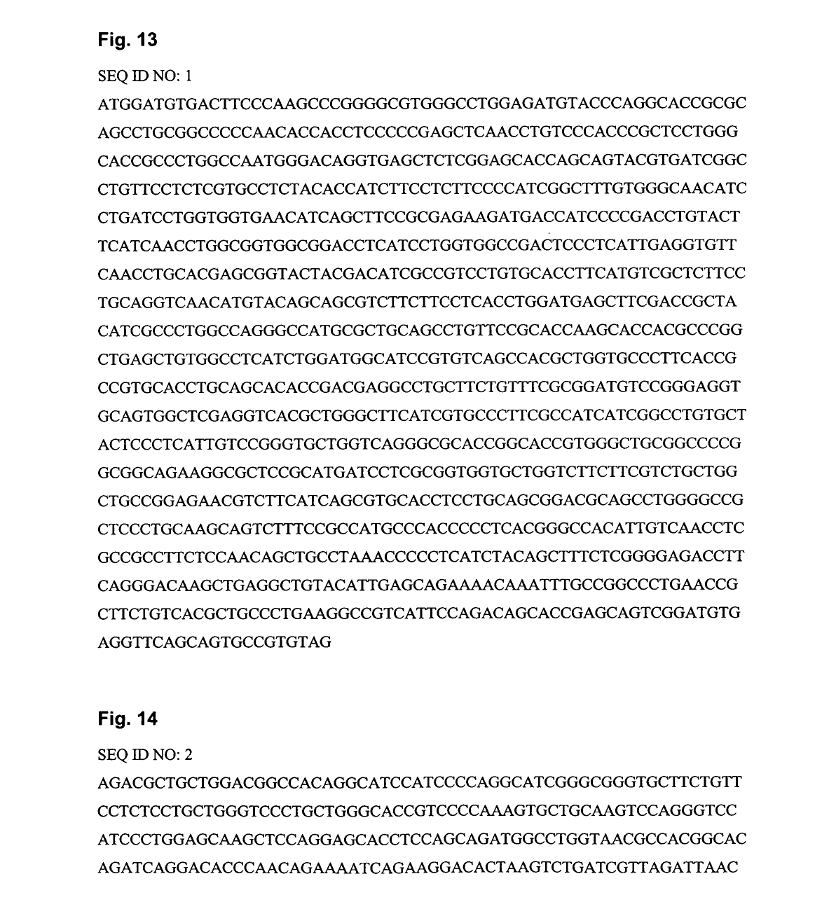

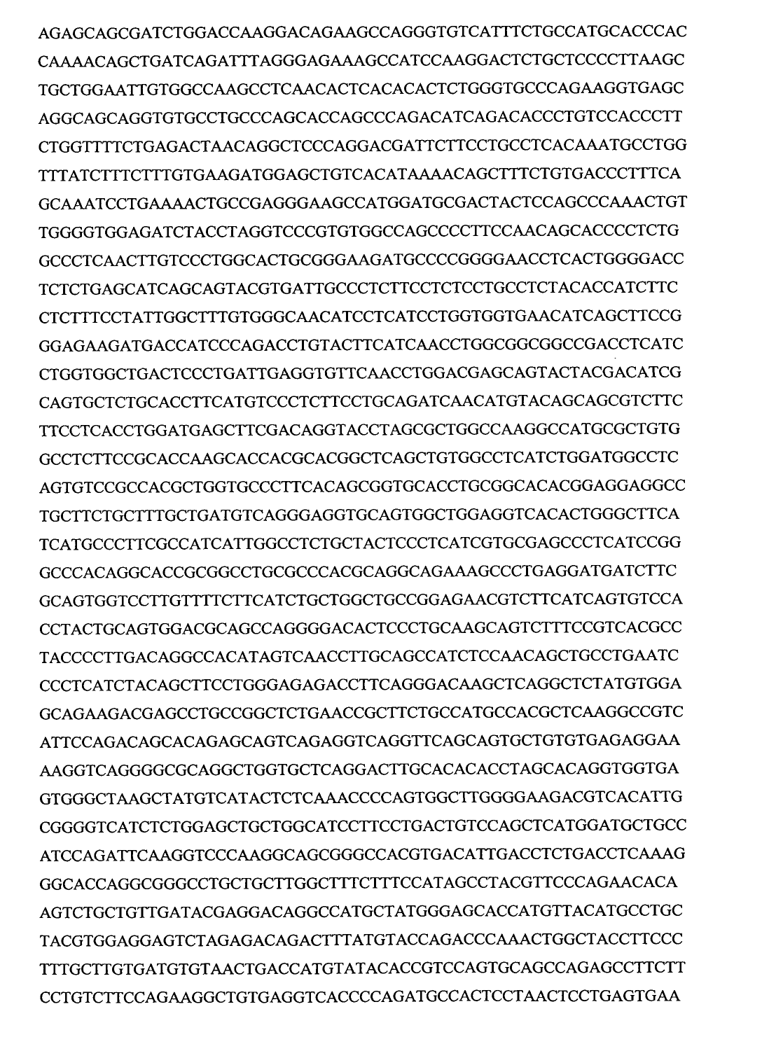

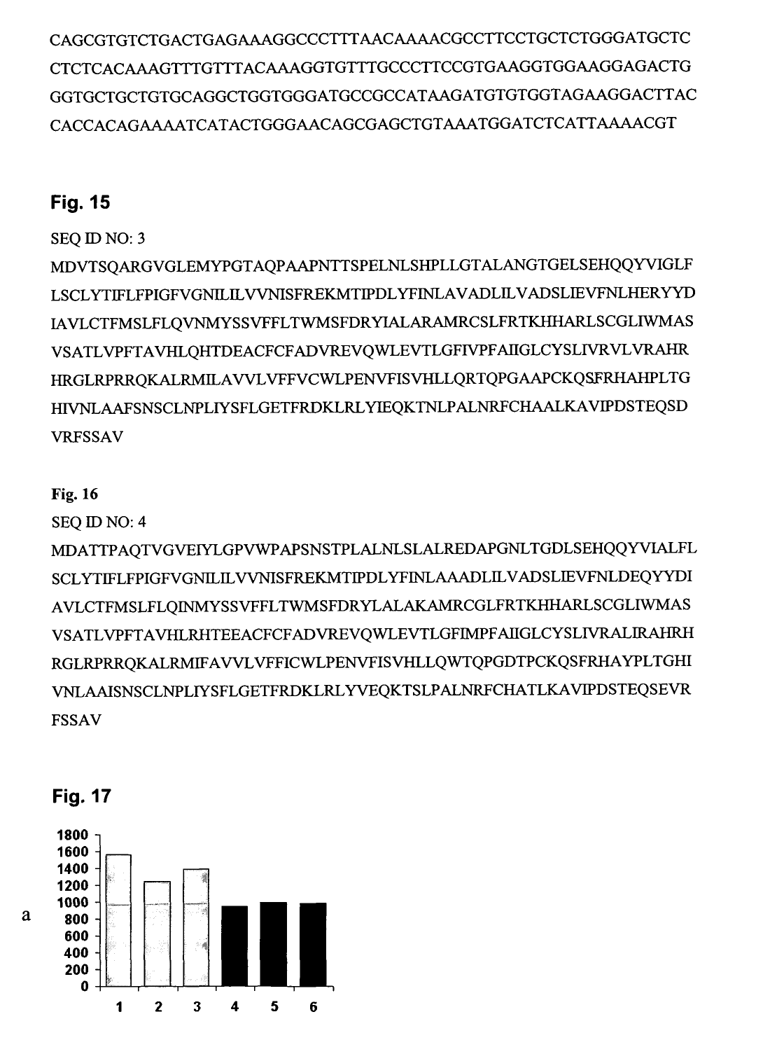

The running distance, top speed, average speed and longest running period of the GPR30 knockout mice was significantly low as compared with those of the wildtype mice (FIGS. 9-12).

### Example 6

### Screening of Compounds for Amelioration Of Cardiac Function Using the GPR30 Knockout Mouse

The GPR30 knockout mouse in the present invention exhibits cardiac dysfunction. The cardiac dysfunction as used herein includes, for example, an increase in left ventricular enddiastolic pressure (LVEDP) and left ventricular relaxation time constant (tau) as well as a decrease in cardiac contractility (LVdPdtₘₐₓ) and maximum velocity of the left ventricular pressure fall (LVdPdtₘᵢₙ). Compounds with efficacy on cardiac function can be screened in GPR30 knockout mice. For example, the compound to be screened can be acutely or chronically administered at various concentrations by parenteral injection, infusion, ingestion, oral administration and other suitable methods in admixture with a pharmaceutically acceptable carrier. A significant decrease in LVEDP and/or tau and/or a significant increase in LVdPdtₘₐₓ and/or LVdPdtₘᵢₙ of the GPR30 knockout mice by a screened compound is indicative that this compound exhibit benefitial properties in other animals and humans with cardiac dysfunction.

### Example 7

### Production of GPR30 Specific Antibodies

Two approaches are utilized to raise antibodies to GPR30, and each approach is useful for generating either polyclonal or monoclonal antibodies. In one approach, denatured protein from reverse phase HPLC separation is obtained in quantities up to 75 mg. This denatured protein is used to immunize mice or rabbits using standard protocols; about 100 μg are adequate for immunization of a mouse, while up to 1 mg might be used to immunize a rabbit. For identifying mouse hybridomas, the denatured protein is radioiodinated and used to screen potential murine B-cell hybridomas for those which produce antibody. This procedure requires only small quantities of protein, such that 20 mg is sufficient for labeling and screening of several thousand clones.

In the second approach, the amino acid sequence of an appropriate GPR30 domain, as deduced from translation of the cDNA, is analyzed to determine regions of high antigenicity. Oligopeptides comprising appropriate hydrophilic regions are synthesized and used in suitable immunization protocols to raise antibodies. The optimal amino acid sequences for immunization are usually at the C-terminus, the N-terminus and those intervening, hydrophilic regions of the polypeptide which are likely to be exposed to the external environment when the protein is in its natural conformation.

Typically, selected peptides, about 15 residues in length, are synthesized using an Applied Biosystems Peptide Synthesizer Model 431A using fmoc-chemistry and coupled to keyhole limpet hemocyanin (KLH; Sigma, St. Louis, Mo.) by reaction with M-maleimidobenzoyl-N-hydroxysuccinimide ester, MBS. If necessary, a cysteine is introduced at the N-terminus of the peptide to permit coupling to KLH. Rabbits are immunized with the peptide-KLH complex in complete Freund's adjuvant. The resulting antisera are tested for antipeptide activity by binding the peptide to plastic, blocking with 1% bovine serum albumin, reacting with antisera, washing and reacting with labeled (radioactive or fluorescent), affinity purified, specific goat anti-rabbit IgG.

Hybridomas are prepared and screened using standard techniques. Hybridomas of interest are detected by screening with labeled GPR30 to identify those fusions producing the monoclonal antibody with the desired specificity. In a typical protocol, wells of plates (FAST; Becton-Dickinson, Palo Alto, Calif.) are coated during incubation with affinity purified, specific rabbit anti-mouse (or suitable antispecies 1 g) antibodies at 10 mg/ml. The coated wells are blocked with 1% bovine serum albumin, (BSA), washed and incubated with supernatants from hybridomas. After washing the wells are incubated with labeled GPR30 at 1 mg/ml. Supernatants with specific antibodies bind more labeled GPR30 than is detectable in the background. Then clones producing specific antibodies are expanded and subjected to two cycles of cloning at limiting dilution. Cloned hybridomas are injected into pristane-treated mice to produce ascites, and monoclonal antibody is purified from mouse ascitic fluid by affinity chromatography on Protein A. Monoclonal antibodies with affinities of at least

10⁸ M⁻¹, preferably 10⁹ to 10¹⁰ M⁻¹ or stronger, are typically made by standard procedures.

### Example 8

### Diagnostic Test Using GPR30 Specific Antibodies

Particular GPR30 antibodies are useful for investigating signal transduction and the diagnosis of infectious or hereditary conditions which are characterized by differences in the amount or distribution of GPR30 or downstream products of an active signaling cascade.

Diagnostic tests for GPR30 include methods utilizing antibody and a label to detect GPR30 in human body fluids, membranes, cells, tissues or extracts of such. The polypeptides and antibodies of the present invention are used with or without modification. Frequently, the polypeptides and antibodies are labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, chromogenic agents, magnetic particles and the like.

A variety of protocols for measuring soluble or membrane-bound GPR30, using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site monoclonal-based immunoassay utilizing monoclonal antibodies reactive to two non-interfering epitopes on GPR30 is preferred, but a competitive binding assay may be employed.

### Example 9

### Purification of Native GPR30 Using Specific Antibodies

Native or recombinant GPR30 is purified by immunoaffinity chromatography using antibodies specific for GPR30. In general, an immunoaffinity column is constructed by covalently coupling the anti-TRH antibody to an activated chromatographic resin.

Polyclonal immunoglobulins are prepared from immune sera either by precipitation with ammonium sulfate or by purification on immobilized Protein A (Pharmacia LKB Biotechnology, Piscataway N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated Sepharose (Pharmacia LKB Biotechnology). The antibody is coupled to the resin, the resin is blocked, and the derivative resin is washed according to the manufacturer's instructions.

Such immunoaffmity columns are utilized in the purification of GPR30 by preparing a fraction from cells containing GPR30 in a soluble form. This preparation is derived by solubilization of whole cells or of a subcellular fraction obtained via differential centrifugation (with or without addition of detergent) or by other methods well known in the art. Alternatively, soluble GPR30 containing a signal sequence is secreted in useful quantity into the medium in which the cells are grown.

A soluble GPR30-containing preparation is passed over the immunoaffmity column, and the column is washed under conditions that allow the preferential absorbance of GPR30 (e.g., high ionic strength buffers in the presence of detergent). Then, the column is eluted under conditions that disrupt antibody/protein binding (e.g., a buffer of pH 2-3 or a high concentration of a chaotrope such as urea or thiocyanate ion), and GPR30 is collected.

### Example 10

### Drug Screening

This invention is particularly useful for screening therapeutic compounds by using GPR30 or binding fragments thereof in any of a variety of drug screening techniques. As GPR30 is a G protein coupled receptor any of the methods commonly used in the art may potentially be used to identify GPR30 ligands. For example, the activity of a G protein coupled receptor such as GPR30 can be measured using any of a variety of appropriate functional assays in which activation of the receptor results in an observable change in the level of some second messenger system, such as adenylate cyclase, guanylylcyclase, calcium mobilization, or inositol phospholipid hydrolysis. Alternatively, the polypeptide or fragment employed in such a test is either free in solution, affixed to a solid support, borne on a cell surface or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. Such cells, either in viable or fixed form, are used for standard binding assays.

Measured, for example, is the formation of complexes between GPR30 and the agent being tested. Alternatively, one examines the diminution in complex formation between GPR30 and a ligand caused by the agent being tested.

Thus, the present invention provides methods of screening for drug canditates, drugs, or any other agents which affect signal transduction. These methods, well known in the art, comprise contacting such an agent with GPR30 polypeptide or a fragment thereof and assaying (i) for the presence of a complex between the agent and GPR30 polypeptide or fragment, or (ii) for the presence of a complex between GPR30 polypeptide or fragment and the cell. In such competitive binding assays, the GPR30 polypeptide or fragment is typically labeled. After suitable incubation, free GPR30 polypeptide or fragment is separated from that present in bound form, and the amount of free or uncomplexed label is a measure of the ability of the particular agent to bind to GPR30 or to interfere with the GPR30-agent complex.

Another technique for drug screening provides high throughput screening for compounds having suitable binding affinity to GPR30 polypeptides. Briefly stated, large numbers of different small peptide test compounds are synthesized on a solid substrate, such as plastic pins or some other surface. The peptide test compounds are reacted with GPR30 polypeptide and washed. Bound GPR30 polypeptide is then detected by methods well known in the art. Purified GPR30 are also coated directly onto plates for use in the aforementioned drug screening techniques. In addition, non-neutralizing antibodies are used to capture the peptide and immobilize it on the solid support.

This invention also contemplates the use of competitive drug screening assays in which neutralizing antibodies capable of binding GPR30 specifically compete with a test compound for binding to GPR30 polypeptides or fragments thereof. In this manner, the antibodies are used to detect the presence of any peptide which shares one or more antigenic determinants with GPR30.

### Example 11

### Rational Drug Design

The goal of rational drug design is to produce structural analogs of biologically active polypeptides of interest or of small molecules with which they interact, agonists, antagonists, or inhibitors. Any of these examples are used to fashion drugs which are more active or stable forms of the polypeptide or which enhance or interfere with the function of a polypeptide in vivo.

In one approach, the three-dimensional structure of a protein of interest, or of a protein-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide is gained by modeling based on the structure of homologous proteins. In both cases, relevant structural information is used to design efficient inhibitors. Useful examples of rational drug design include molecules which have improved activity or stability or which act as inhibitors, agonists, or antagonists of native peptides.

It is also possible to isolate a target-specific antibody, selected by functional assay, as described above, and then to solve its crystal structure. This approach, in principle, yields a pharmacore upon which subsequent drug design is based. It is possible to bypass protein crystallography altogether by generating anti-idiotypic antibodies (anti-ids) to a functional, pharmacologically active antibody. As a mirror image of a mirror image, the binding site of the anti-ids is expected to be an analog of the original receptor. The anti-id is then used to identify and isolate peptides from banks of chemically or biologically produced peptides. The isolated peptides then act as the pharmacore.

By virtue of the present invention, sufficient amount of polypeptide are made available to perform such analytical studies as X-ray crystallography. In addition, knowledge of the GPR30 amino acid sequence provided herein provides guidance to those employing computer modeling techniques in place of or in addition to x-ray crystallography.

### Example 12

### Identification of Other Members of the Signal Transduction Complex

The inventive purified GPR30 is a research tool for identification, characterization and purification of interacting G or other signal transduction pathway proteins. Radioactive labels are incorporated into a selected GPR30 domain by various methods known in the art and used in vitro to capture interacting molecules. A preferred method involves labeling the primary amino groups in GPR30 with ¹²⁵I Bolton-Hunter reagent. This reagent has been used to label various molecules without concomitant loss of biological activity.

Labeled GPR30 is useful as a reagent for the purification of molecules with which it interacts. In one embodiment of affinity purification, membrane-bound GPR30 is covalently coupled to a chromatography column. Cell-free extract derived from synovial cells or putative target cells is passed over the column, and molecules with appropriate affinity bind to GPR30. GPR30-complex is recovered from the column, and the GPR30-binding ligand disassociated and subjected to N-terminal protein sequencing. The amino acid sequence information is then used to identify the captured molecule or to design degenerate oligonucleotide probes for cloning the relevant gene from an appropriate cDNA library.

In an alternate method, antibodies are raised against GPR30, specifically monoclonal antibodies. The monoclonal antibodies are screened to identify those which inhibit the binding of labeled GPR30. These monoclonal antibodies are then used therapeutically.

### Example 13

### Use and Administration of Antibodies or Agonists

Antibodies or agonists of GPR30 or other treatments and compounds that are limiters of signal transduction (LSTs), provide different effects when administered therapeutically. LSTs are formulated in a nontoxic, inert, pharmaceutically acceptable aqueous carrier medium preferably at a pH of about 5 to 8, more preferably 6 to 8, although pH may vary according to the characteristics of the antibody or agonist being formulated and the condition to be treated. Characteristics of LSTs include solubility of the molecule, its half-life and antigenicity/immunogenicity. These and other characteristics aid in defining an effective carrier. Native human proteins are preferred as LSTs, but organic or synthetic molecules resulting from drug screens are equally effective in particular situations.

LSTs are delivered by known routes of administration including but not limited to topical creams and gels; transmucosal spray and aerosol; transdermal patch and bandage; injectable, intravenous and lavage formulations; and orally administered liquids and pills particularly formulated to resist stomach acid and enzymes. The particular formulation, exact dosage, and route of administration are determined by the attending physician and varies according to each specific situation.

Such determinations are made by considering multiple variables such as the condition to be treated, the LST to be administered, and the pharmacokinetic profile of a particular LST. Additional factors which are taken into account include severity of the disease state, patient's age, weight, gender and diet, time and frequency of LST administration, possible combination with other drugs, reaction sensitivities, and tolerance/response to therapy. Long acting LST formulations might be administered every 3 to 4 days, every week, or once every two weeks depending on half-life and clearance rate of the particular LST.

Normal dosage amounts vary from 0.1 to 10⁵ μg, up to a total dose of about 1 g, depending upon the route of administration. Guidance as to particular dosages and methods of delivery is provided in the literature; see U.S. Pat. Nos. 4,657,760; 5,206,344; or 5,225,212. Those skilled in the art employ different formulations for different LSTs. Administration to cells such as nerve cells necessitates delivery in a manner different from that to other cells such as vascular endothelial cells.

It is contemplated that abnormal signal transduction, trauma, or diseases which trigger GPR30 activity are treatable with LSTs. These conditions or diseases are specifically diagnosed by the tests discussed above, and such testing should be performed in suspected cases of viral, bacterial or fungal infections, allergic responses, mechanical injury associated with trauma, hereditary diseases, lymphoma or carcinoma, or other conditions which activate the genes of lymphoid or neuronal tissues.

### Example 14

### Microarray Experiments

Total RNA extracted from cardiac tissue and was purified using an affinity resin column (RNeasy; Qiagen, Hilden, Germany), quantified by spectrophotometry (absorbance 260 nm), and the quality of RNA was assessed by microfluidics electrophoretical separation with a Bioanalyzer (Agilent Technologies, Palo Alto, USA). Purified total RNA (1μg) was converted to cDNA using the Superscript Choice cDNA synthesis kit (Invitrogen, Carlsbad, Calif., USA), incorporating a T7-(dT)24 primer. Double-stranded cDNA was then purified by affinity resin column (Clean up Kit, Qiagen, Hilden, Germany) with ethanol extraction. Purified cDNA was used as a template for in vitro transcription reaction for the synthesis of biotinylated cRNA using an Enzo BioArray HighYield RNA transcription labeling kit (Affymetrix, Santa Clara, Calif.), and further purified using an affinity resin column (Clean up Kit, Qiagen, Hilden, Germany). After purification, in vitro cRNA was fragmented in buffer containing magnesium at 95° C. for 35 min. Fragmented cRNA was hybridized onto the Affymetrix GeneChip Human Genome U133 Plus 2.0 Array. Briefly, 15 pig fragmented cRNA was added along with control cRNA (BioB, BioC, and BioD), herring sperm DNA (10 mg/ml), 10% DMSO, and acetylated BSA (50 mg/ml) to the hybridization buffer. The hybridization mixture was heated at 99° C. for 5 min, incubated at 45° C. for 5 min, centrifuged for 5 min at 13,000 rpm, and injected into the microarray. After hybridization at 45° C. for 16 h rotating at 60 rpm, the array was washed and stained with the Affymetrix Fluidics Protocols-antibody amplification for Eukaryotic Targets, and scanned using an Affymetrix microarray scanner (GeneChip Scanner 3000 7G system) at 570 nm.

### Example 15

Data analysis from microarray experiments

Raw data analysis and scaling were performed in Microarray Suite 5.0 software (Affymetrix), and normalization and further analysis in expressionist Pro 3.0 (Genedata). Results for HG-U133 Plus 2.0 arrays were subjected to global scaling with a target intensity of 50.

Base-2 logarithms were calculated for all expression values and taken for subsequent statistical analysis to analyze the differential expression between the two groups.

### Example 16

### Screening of Agonists of GPR30 for Amelioration of Cardiac Function

Agonists of GPR30 can be screened in animal models of cardiomyopathy. Animal models of cardiomyopathy include but are not limited to the Bio 14.6 hamster, cardiac hypertrophy or heart failure induced by various manipulations such as coronary artery ligation, drugs, pressure and/or volume overload and chronic rapid pacing in mice, rats, dogs, rabbits and many other animals as well as transgenic mice which exhibit cardiomyopathy. For example, the agonists of GPR30 to be screened can be acutely or chronically administered at various concentrations by parenteral injection, infusion, ingestion, oral administration and other suitable methods in admixture with a pharmaceutically acceptable carrier. A significant decrease in LVEDP and/or tau and/or a significant increase in LVdPdtₘₐₓ and/or LVdPdtₘᵢₙ by a screened agonist og GPR30 is indicative that this compound exhibit benefitial properties in other animals and humans with cardiac dysfunction.

### REFERENCES

U.S. 4,522,811
WO 84/03564
WO 92/01810
Ahuja et al., 1993, J Biol Chem 268: 20691-20694
Belmadan et al., 2007, Am J Pathol. 171: 777-789
Carmeci et al., 1997, Genomics 45: 607-617
Civelli, 2005, Trends Pharmacol. Sci. 26: 15-19
Curci et al., 1998, Clin. Invest. 102: 1900-1910
Date et al., 2000, Nippon Rinsho 58: 233-239
Dobner et al., 1992, Eur J Immunol 22 : 2795-2799
Felkin et al., 2006, J Heart Lung Transplant. 25: 1413-1419
Feng and Gregor, 1997, Biochem. Biophys. Res. Commun. 231: 651-654
Feng et al., 2007, Gene 403: 170-177
De Fraipont et al., 2000, Endocr. Metab. 85: 4734-4741
Gyorke et al. 2008, Cardiovasc Res 77 : 245-255
Haseloff et al., 1988 , Nature 334: 585-591
Iwabuchi et al., 1993, Oncogene 8: 1693-1696
Jiang et al, 1995, Oncogene 11: 2477-2486
Kirchhefer, 2001, J. Biol. Chem. 276: 4142-4149
Kubota, 1997, Circ Res. 81: 627-635
Lam, 1997, Anticancer Drug Res. 12: 145-67
Lefkowitz, 1991, Nature 351: 353-354
McConnell et al., 1992 , Science 257: 1906-1912
O'Dowd et al., 1997, Genomics 47: 310-313
Owman et al., 1996, Biochem. Biophys. Res. Commun. 228: 285-292
Polyakova et al., 2008, J Cell Mol Med. 12:189-208
Rezgui et al., 2005, J Biol Chem. 280: 39302-39308
Rouault et al., 1996, Nature Genet. 14: 482-486
Salazar et al., 2007, Biochem Biophy Acta 1768: 1006-1018
Schuster et al., 1992, Eur J Pediatr 151: 718-725
Sjolander and Urbaniczky, 1991, Anal. Chem. 63: 2338-2345
Szabo et al., 1995, Curr. Opin. Struct. Biol. 5: 699-705
Thakar et al., 2005, J. Clin. Invest. 115: 3451-3459
Terentyev et al., 2005, Circ Res. 96: 651-8
Thomas et al., 2005, Endocrinology 146: 624-632
Volpert et al., 2002, Cancer Cell 2: 473-483
Vila et al., 2007, Int J Cardiol. August 27
Vila et al., 2008, Thromb Res. 121: 611-615
Zang et al., 1997, J Biol Chem. 272: 23389-23397
Zhanq et al., 2001, Am J Physiol. 280: H1782-111792

1. A transgenic non-human animal which is deficient in expressing GPR30.

2. The transgenic non-human animal of claim 1, wherein the animal exhibits a cardiovascular defects.

3. The transgenic non-human animal of claim 2, wherein the cardiovascular defect is characterized by an increase in LVEDP and tau and a decrease in LVdPdtₘₐₓ and LVdPdtₘᵢₙ.

4. A transgenic non-human animal of claims 1, wherein the animal is a rodent.

5. A method for selecting a potential therapeutic agent for treating the cardiovascular defects occurring in the animal of claim 2 comprising:
i) administering one ore more agents to be tested to the animal of claim 2, and
ii) determining whether said cardiovascular defects occurring in said animal have changed as a result of administration of said agent or agents.

6. A cell isolated from the transgenic non-human animal of claim 1.

7. A cell line derived from a cell according to claim 6.

8. A method of screening for therapeutic agents useful in the treatment of cardiovascular disease in a mammal comprising the steps of
i) contacting a test compound with a GPR30 polypeptide,
ii) detecting binding of said test compound to said GPR30 polypeptide,
iii) selecting an agent as useful in the treatment of cardiovascular disease if it increases the activity of the GPR30 polypeptide.

9. The method of screening of claim 8, further comprising the steps of
i) determining the activity of the GPR30 polypeptide at a certain concentration of the test compound or in the absence of said test compound, and
ii) determining the activity of said polypeptide at a different concentration of said test compound

10. The method of screening of claim 8, wherein
the activity of the GPR30 polypeptide is determined in the presence of a compound known to be a regulator of a GPR30 polypeptide.

11. The method of claim 8, wherein the step of contacting is in or at the surface of a cell.

12. The method of screening of claim 8, wherein the cell is in vitro.

13. The method of screening of claim 8, wherein the polypeptide is coupled to a detectable label.

14. (canceled)

15. (canceled)


SEQUENCE LISTING (4 sequences)

SEQ ID NO: 1 - DNA, 1128 bp, Homo sapiens
atggatgtgacttcccaagcccggggcgtgggcctggagatgtacccaggcaccgcgcagcctgcggcccccaacaccacctcccccgagctcaacctgtcccacccgctcctgggcaccgccctggccaatgggacaggtgagctctcggagcaccagcagtacgtgatcggcctgttcctctcgtgcctctacaccatcttcctcttccccatcggctttgtgggcaacatcctgatcctggtggtgaacatcagcttccgcgagaagatgaccatccccgacctgtacttcatcaacctggcggtggcggacctcatcctggtggccgactccctcattgaggtgttcaacctgcacgagcggtactacgacatcgccgtcctgtgcaccttcatgtcgctcttcctgcaggtcaacatgtacagcagcgtcttcttcctcacctggatgagcttcgaccgctacatcgccctggccagggccatgcgctgcagcctgttccgcaccaagcaccacgcccggctgagctgtggcctcatctggatggcatccgtgtcagccacgctggtgcccttcaccgccgtgcacctgcagcacaccgacgaggcctgcttctgtttcgcggatgtccgggaggtgcagtggctcgaggtcacgctgggcttcatcgtgcccttcgccatcatcggcctgtgctactccctcattgtccgggtgctggtcagggcgcaccggcaccgtgggctgcggccccggcggcagaaggcgctccgcatgatcctcgcggtggtgctggtcttcttcgtctgctggctgccggagaacgtcttcatcagcgtgcacctcctgcagcggacgcagcctggggccgctccctgcaagcagtctttccgccatgcccaccccctcacgggccacattgtcaacctcgccgccttctccaacagctgcctaaaccccctcatctacagctttctcggggagaccttcagggacaagctgaggctgtacattgagcagaaaacaaatttgccggccctgaaccgcttctgtcacgctgccctgaaggccgtcattccagacagcaccgagcagtcggatgtgaggttcagcagtgccgtgtag

SEQ ID NO: 2 - DNA, 2497 bp, Mus musculus
agacgctgctggacggccacaggcatccatccccaggcatcgggcgggtgcttctgttcctctcctgctgggtccctgctgggcaccgtccccaaagtgctgcaagtccagggtccatccctggagcaagctccaggagcacctccagcagatggcctggtaacgccacggcacagatcaggacacccaacagaaaatcagaaggacactaagtctgatcgttagattaacagagcagcgatctggaccaaggacagaagccagggtgtcatttctgccatgcacccaccaaaacagctgatcagatttagggagaaagccatccaaggactctgctccccttaagctgctggaattgtggccaagcctcaacactcacacactctgggtgcccagaaggtgagcaggcagcaggtgtgcctgcccagcaccagcccagacatcagacaccctgtccacccttctggttttctgagactaacaggctcccaggacgattcttcctgcctcacaaatgcctggtttatctttctttgtgaagatggagctgtcacataaaacagctttctgtgaccctttcagcaaatcctgaaaactgccgagggaagccatggatgcgactactccagcccaaactgttggggtggagatctacctaggtcccgtgtggccagccccttccaacagcacccctctggccctcaacttgtccctggcactgcgggaagatgccccggggaacctcactggggacctctctgagcatcagcagtacgtgattgccctcttcctctcctgcctctacaccatcttcctctttcctattggctttgtgggcaacatcctcatcctggtggtgaacatcagcttccgggagaagatgaccatcccagacctgtacttcatcaacctggcggcggccgacctcatcctggtggctgactccctgattgaggtgttcaacctggacgagcagtactacgacatcgcagtgctctgcaccttcatgtccctcttcctgcagatcaacatgtacagcagcgtcttcttcctcacctggatgagcttcgacaggtacctagcgctggccaaggccatgcgctgtggcctcttccgcaccaagcaccacgcacggctcagctgtggcctcatctggatggcctcagtgtccgccacgctggtgcccttcacagcggtgcacctgcggcacacggaggaggcctgcttctgctttgctgatgtcagggaggtgcagtggctggaggtcacactgggcttcatcatgcccttcgccatcattggcctctgctactccctcatcgtgcgagccctcatccgggcccacaggcaccgcggcctgcgcccacgcaggcagaaagccctgaggatgatcttcgcagtggtccttgttttcttcatctgctggctgccggagaacgtcttcatcagtgtccacctactgcagtggacgcagccaggggacactccctgcaagcagtctttccgtcacgcctaccccttgacaggccacatagtcaaccttgcagccatctccaacagctgcctgaatcccctcatctacagcttcctgggagagaccttcagggacaagctcaggctctatgtggagcagaagacgagcctgccggctctgaaccgcttctgccatgccacgctcaaggccgtcattccagacagcacagagcagtcagaggtcaggttcagcagtgctgtgtgagaggaaaaggtcaggggcgcaggctggtgctcaggacttgcacacacctagcacaggtggtgagtgggctaagctatgtcatactctcaaaccccagtggcttggggaagacgtcacattgcggggtcatctctggagctgctggcatccttcctgactgtccagctcatggatgctgccatccagattcaaggtcccaaggcagcgggccacgtgacattgacctctgacctcaaagggcaccaggcgggcctgctgcttggctttctttccatagcctacgttcccagaacacaagtctgctgttgatacgaggacaggccatgctatgggagcaccatgttacatgcctgctacgtggaggagtctagagacagactttatgtaccagacccaaactggctaccttccctttgcttgtgatgtgtaactgaccatgtatacaccgtccagtgcagccagagccttcttcctgtcttccagaaggctgtgaggtcaccccagatgccactcctaactcctgagtgaacagcgtgtctgactgagaaaggccctttaacaaaacgccttcctgctctgggatgctcctctcacaaagtttgtttacaaaggtgtttgcccttccgtgaaggtggaaggagactgggtgctgctgtgcaggctggtgggatgccgccataagatgtgtggtagaaggacttaccaccacagaaaatcatactgggaacagcgagctgtaaatggatctcattaaaacgt

SEQ ID NO: 3 - protein, 375 aa, Homo sapiens
MDVTSQARGVGLEMYPGTAQPAAPNTTSPELNLSHPLLGTALANGTGELSEHQQYVIGLFLSCLYTIFLFPIGFVGNILILVVNISFREKMTIPDLYFINLAVADLILVADSLIEVFNLHERYYDIAVLCTFMSLFLQVNMYSSVFFLTWMSFDRYIALARAMRCSLFRTKHHARLSCGLIWMASVSATLVPFTAVHLQHTDEACFCFADVREVQWLEVTLGFIVPFAIIGLCYSLIVRVLVRAHRHRGLRPRRQKALRMILAVVLVFFVCWLPENVFISVHLLQRTQPGAAPCKQSFRHAHPLTGHIVNLAAFSNSCLNPLIYSFLGETFRDKLRLYIEQKTNLPALNRFCHAALKAVIPDSTEQSDVRFSSAV

SEQ ID NO: 4 - protein, 375 aa, mus musculus
MDATTPAQTVGVEIYLGPVWPAPSNSTPLALNLSLALREDAPGNLTGDLSEHQQYVIALFLSCLYTIFLFPIGFVGNILILVVNISFREKMTIPDLYFINLAAADLILVADSLIEVFNLDEQYYDIAVLCTFMSLFLQINMYSSVFFLTWMSFDRYLALAKAMRCGLFRTKHHARLSCGLIWMASVSATLVPFTAVHLRHTEEACFCFADVREVQWLEVTLGFIMPFAIIGLCYSLIVRALIRAHRHRGLRPRRQKALRMIFAVVLVFFICWLPENVFISVHLLQWTQPGDTPCKQSFRHAYPLTGHIVNLAAISNSCLNPLIYSFLGETFRDKLRLYVEQKTSLPALNRFCHATLKAVIPDSTEQSEVRFSSAV